(19) United States
(12) Patent Application Publication
(10) Pub. No.: US 2024/0050763 A1
(43) Pub. Date: Feb. 15, 2024

(54) ELECTROENCEPHALOGRAM (EEG) BASED TRANSCRANIAL MAGNETIC STIMULATION (TMS) NETWORKS

(71) Applicant: WAVE NEUROSCIENCE, INC., Newport Beach, CA (US)

(72) Inventors: James William PHILLIPS, Fountain Valley, CA (US); Alexander RING, Newport Beach, CA (US)

(73) Assignee: WAVE NEUROSCIENCE, INC., Newport Beach, CA (US)

(21) Appl. No.: 18/280,883

(22) PCT Filed: Mar. 25, 2022
(86) PCT No.: PCT/US22/71358

§ 371 Date: Sep. 7, 2023

Related U.S. Application Data
(60) Provisional application No. 63/166,944, filed on Mar. 26, 2021.

(51) Int. Cl. A61N 2/00; G16H 20/40; G16H 10/60; G16H 70/20; A61N 2/02

(52) U.S. Cl. CPC A61N 2/006; G16H 20/40; G16H 10/60; G16H 70/20; A61N 2/02

(57) ABSTRACT

A networked system for providing transcranial magnetic stimulation (TMS) based on analyzed electroencephalogram (EEG) data is disclosed. The networked system interfaces with portable neuro¬electroencephalogram synchronization therapy (NEST) devices for capturing EEG data from a patient. The network also may also include an electrophysiology database and customized TMS treatment system for receiving the EEG data from the portable NEST system. The electrophysiology database and customized TMS treatment system are configurable and operable to determine a TMS treatment based on the received EEG data. The portable NEST system may provide synchronized TMS based on the determined TMS treatment.

### CROSS-REFERENCE

This application claims the benefit of U.S. Provisional Application No. 63/166,944, filed Mar. 26, 2021, entitled METHODS, SYSTEMS, KITS AND APPARATUSES FOR PROVIDING TRANSCRANIAL MAGNETIC STIMULATION (TMS) BASED ON ELECTROENCEPHALOGRAM (EEG) DATA, which application is incorporated herein in its entirety by reference.

This application is related to the following co-pending patent applications: application Serial No. This application is related to the following co-pending patent applications: application Serial No. PCT/US22/71355 filed Mar. 25, 2022; and application Serial No. PCT/US22/71357 filed Mar. 25, 2022, which are incorporated herein in their entirely by reference.

### BACKGROUND

### Field

The disclosure relates to networks and systems for providing transcranial magnetic stimulation (TMS) therapy based on electroencephalogram (EEG) data.

### Background

Mental disorders cause serious problems for affected people, their families, and society. Currently, psychiatrists and neurophysiologists treat these disorders with a variety of medications, many of which have significant negative side effects. Mental disorders can be painful, debilitating, and very costly for the affected individual and their family. Approximately one in five adults in the US experiences a mental disorder in a given year. 18.1% of adults in the US experience an anxiety disorder, such as posttraumatic stress disorder, obsessive-compulsive disorder (OCD) and specific phobias. 6.9% of adults in the US have at least one major depressive episode each year. 1.1% of adults in the US live with schizophrenia. In fact, mental disorders are the third most common cause of hospitalization in the US for both youth and adults aged 18-44 and the consequences of lack of treatment are significant. Sadly, suicide is the 10th leading cause of death in the U.S. and the 2nd leading cause of death for those aged 15-24. Each day, approximately 18-22 veterans die by suicide.

A key factor in treatment of mental disorders is proper diagnosis. The standard method of diagnosing mental disorders has been with either the Diagnostic and Statistical Manual of Mental Disorders (DSM) or the International Statistical Classification of Diseases and Related Health Problems (ICD), Chapter 5: Mental and behavioral disorders. Both standards primarily involve diagnosis using conversation with the patient regarding symptoms and behavior. This standard has the disadvantage of being subjectively based on the interviewer's perception, which lessens the diagnostic reliability, sometimes resulting in two clinicians arriving at different diagnoses for the same patient. Additionally, a patient's responses to questions may vary based upon their present situation or life circumstance. Because the DSM and ICD are primarily concerned with the signs and symptoms of mental disorders, rather than the underlying causes, there is a general lack of pathophysiological understanding of mental disorders.

It is apparent that a repeatable and reliable system for the diagnosis of mental disorders that is based on measurable data, independent of the biases and interpretation of an interviewer or a fluctuating situational condition of the patient, would provide significant benefit to patients and to the psychiatric community.

Treatment of these disorders with magnetic fields, such as those generated by transcranial magnetic stimulation (TMS), may generate positive therapeutic responses.

TMS is a generally known non-invasive procedure that uses magnetic pulses to stimulate nerve cells and neuronal circuitry in the brain to improve mental disorders such as depression. Magnetic pulses delivered at regular intervals, described as repetitive magnetic pulses, are referred to as rTMS. Traditionally, coils (e.g., a large electric coil) have been used to generate rTMS. The large electric coil (e.g., electromagnetic coil) may be placed against a patient's scalp when applying stimulation. Some studies have shown that rTMS can reduce the negative symptoms of schizophrenia, obsessive compulsive disorder (OCD), and depression under certain circumstances.

Magnetic fields, especially time and magnitude-varying magnetic fields, can also be generated by movement of one or more magnets. For example, some devices provide TMS by using rotating permanent magnets where magnets are positioned around a patient's head to provide TMS towards regions of the patient's brain to stimulate nerve cells in those specific regions of the brain. In some of these examples, rTMS uses an electromagnet placed on the scalp that generates a series of magnetic field pulses roughly the strength of an MRI scan.

There are also normative electroencephalogram (EEG) databases that have been in existence for several years at a few universities. The normative EEG databases are typically accompanied by clinical scores or evaluations of some kind for EEG data that was collected from patients. These normative EEG databases have been used to aid medical professionals in evaluating neurological statuses of patients prior to and after therapy to adjust therapy as needed based on EEG biofeedback. This deeper knowledge can benefit patients, medical professionals (e.g., clinicians), and the greater neurological or neurotherapy field. These databases are usually based on a wide age range from children up to adults. Examples of universities having these normative EEG databases include New York University (NYU) and the University of Maryland.

What is needed are networks and systems that facilitate delivery of customize TMS based on normative EEG data.

### SUMMARY

The disclosure relates generally to networks and system for providing transcranial magnetic stimulation (TMS) based on normative electrophysiological measurement such as electroencephalogram (EEG) data. The normative database allows the systems and methods to assess deviations from expected normal parameters in a population.

A network integrated with devices for providing TMS based on normative EEG data is disclosed. The devices include: a portable neuro-electroencephalogram synchronization therapy (NEST) system for capturing the EEG data from a patient in communication with a normative EEG database which is operable to customize the application of TMS based on the EEG data compared to the normative EEG data from the database. A TMS treatment system can receive EEG data from the portable NEST system. The electrophysiology database and customized TMS treatment system may determine a TMS treatment based on the received EEG data. The portable NEST system may provide synchronized transcranial magnetic stimulation based on the determined TMS treatment.

In some embodiments, the EEG data may include EEGs from before and after use of the portable NEST system to indicate a set of effects of the TMS treatment. The EEG data may be stored in an object storage database as a set of objects.

The portable NEST system may also provide the synchronized transcranial magnetic stimulation relating to a defined treatment protocol delivered by the network.

In some embodiments, the network may have internet of things (IoT) capabilities when connected to a network.

The electrophysiology database and customized TMS treatment system may be a customized TMS treatment platform. Additionally, the electrophysiology database and customized TMS treatment system may include a customized treatment software application for providing the TMS determined treatments based on the EEG data. The electrophysiology database and customized TMS treatment system may also provide a report of a brain health indicator based on the EEG data and/or a report providing a diagnosis and a recommended determined treatment based on the EEG data.

Artificial intelligence can also be used for determining a diagnosis and/or determining and recommending the treatment for the patient.

Additionally, the electrophysiology database and customized TMS treatment system may provide a set of aggregated data analytics on the EEG data from a set of multiple patients that may be used as an input for at least one of determining a diagnosis and recommending a treatment for the patient. The electrophysiology database and customized TMS treatment system may generate predictive analytics based on the aggregated data analytics that may be used to identify patients and provide appropriate treatment for the identified patients based on the EEG data. Additionally, the electrophysiology database and customized TMS treatment system may apply big data analytics to model various treatments in terms of predicted impacts to patients and results of treatments.

The networks and systems can also recommend TMS based on EEG data. The systems may include an electrophysiology database and a customized TMS treatment system for accessing the EEG data for a patient. The electrophysiology database and customized TMS treatment system may classify the patient as having a particular brain type based on the EEG data, and the electrophysiology database and customized TMS treatment system may determine a TMS treatment based on the classified brain type and the EEG data. The electrophysiology database and customized TMS treatment system may recommend synchronized transcranial magnetic stimulation based on the determined TMS treatment. The electrophysiology data may include patient EEG data from pre and post treatments for providing feedback in terms of diagnosis and treatment of the patient. Patients can be categorized by brain type, e.g. a highly rhythmic brain, a less rhythmic brain, a low energy brain, or a high energy brain. The particular brain type may also be at least one of an absorber-type of personality or an emitter-type of personality.

Systems may include a portable NEST system for capturing the EEG data from the patient that may be accessed by the electrophysiology database and customized TMS treatment system. The portable NEST system may provide the recommended synchronized transcranial magnetic stimulation to the patient. Systems for recommending TMS based on EEG data are disclosed. The systems may include an electrophysiology database and customized TMS treatment system for accessing the EEG data for a patient. The electrophysiology database and customized TMS treatment system may analyze the EEG data to indicate brain health of the patient, and the electrophysiology database and customized TMS treatment system may determine TMS treatment based on the brain health indication and the EEG data. The electrophysiology database and customized TMS treatment system may recommend synchronized transcranial magnetic stimulation based on the determined TMS treatment. EEG data may also include EEG data from pre and post-treatments for providing feedback in terms of diagnosis and treatment of the patient.

An electrophysiology database and customized TMS treatment system may also provide reporting capabilities. Reports include an indication of brain health for the patient. In turn, the brain health indication can be used to assist in diagnosing the patient.

The NEST systems may also provide the recommended synchronized transcranial magnetic stimulation to the patient. The systems may include an electrophysiology database and customized TMS treatment system for accessing the EEG data for a patient. The electrophysiology database and customized TMS treatment system may also provide bursting analysis of the EEG data, and the electrophysiology database and customized TMS treatment system may determine a TMS treatment based on the bursting analysis and the EEG data. The electrophysiology database and customized TMS treatment system may recommend synchronized transcranial magnetic stimulation based on the determined TMS treatment. Additionally, the electrophysiology database and customized TMS treatment system may use the bursting analysis for diagnosing the patient. In some embodiments, the electrophysiology database and customized TMS treatment system may use the bursting analysis for determining treatment parameters.

The NEST system may also be accessed by the electrophysiology database and customized TMS treatment system, and the portable NEST system may provide the recommended synchronized transcranial magnetic stimulation to the patient.

The system may include a portable NEST system for capturing EEG data from a patient. The portable NEST system may have one or more rotating magnets for generating an alternating magnetic field. The portable NEST system may be operable to determine a TMS treatment recommendation based on the EEG data or may receive the TMS treatment recommendation from a second system based on the EEG data. The portable NEST system may also provide synchronized transcranial magnetic stimulation by using the rotating magnets to generate the alternating magnetic field based on the determined TMS treatment or the recommended TMS treatment. The one or more magnets may be diametrically magnetized. Additionally, the one or more magnets may be positioned in a Halbach array and/or positioned to target a specific region of a brain of the patient.

A second system may be provided in communication with the network. Suitable second systems include an electrophysiology database and customized TMS treatment system for receiving the EEG data from the portable NEST system, and the electrophysiology database and customized TMS treatment system may determine the TMS treatment based on the received EEG data.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

U.S. Pat. No. 9,962,555 issued May 8, 2018 to Charles et al.; and U.S. Pat. No. 10,835,754 issued Nov. 17, 2020 to Charles et al.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a diagrammatic environment view that depicts an exemplary portable NEST system and an electrophysiology database and customized TMS treatment system communicating via a network, and communicating with various other systems, devices, processes, and information sources according to embodiments of the disclosure;

FIG. 2A is a diagrammatic view that depicts examples of the portable NEST system of FIG. 1 according to embodiments of the disclosure;

FIG. 2B is a diagrammatic environment view that depicts an exemplary portable NEST system and an electrophysiology database and customized TMS treatment system communicating via a network, and communicating with various other systems, devices, processes, and information sources according to embodiments of the disclosure;

FIG. 3 is a diagrammatic view that depicts details of the electrophysiology database and customized TMS treatment system of FIG. 1 according to embodiments of the disclosure; and

FIG. 4 is a graph view that depicts a reporting screenshot of a burst histogram of a sample EEG according to embodiments of the disclosure.

### DETAILED DESCRIPTION

The disclosure relates to an electroencephalogram (EEG) and transcranial magnetic stimulation (TMS) process. In examples, the EEG and TMS process may be executed by any combination of a neuro-electroencephalogram synchronization therapy (NEST) system, an electrophysiology database and/or a TMS treatment system (optionally using an electrophysiology database and optionally providing for customized, patient-specific TMS treatment protocols, and/or optionally using a combined electrophysiology database and customized TMS treatment system), and/or any other computing device.

The portable NEST system may be easily carried or moved because the device or system may be lighter, compact, transportable, mobile, lightweight and/or smaller than typical or standard devices or systems. The portable NEST systems may include wearable NEST systems, NEST systems configured as headgear, positionable and removable NEST systems (e.g., may be conveniently positioned on or above a chair or seat and then easily removed from the chair or seat), modular NEST systems (e.g., capable of being integrated into another device or system), etc. The portable NEST system may be sized to have a maximum volume or a dimension range, such as about 285 mm length by about 194 mm width by about 188 mm height.

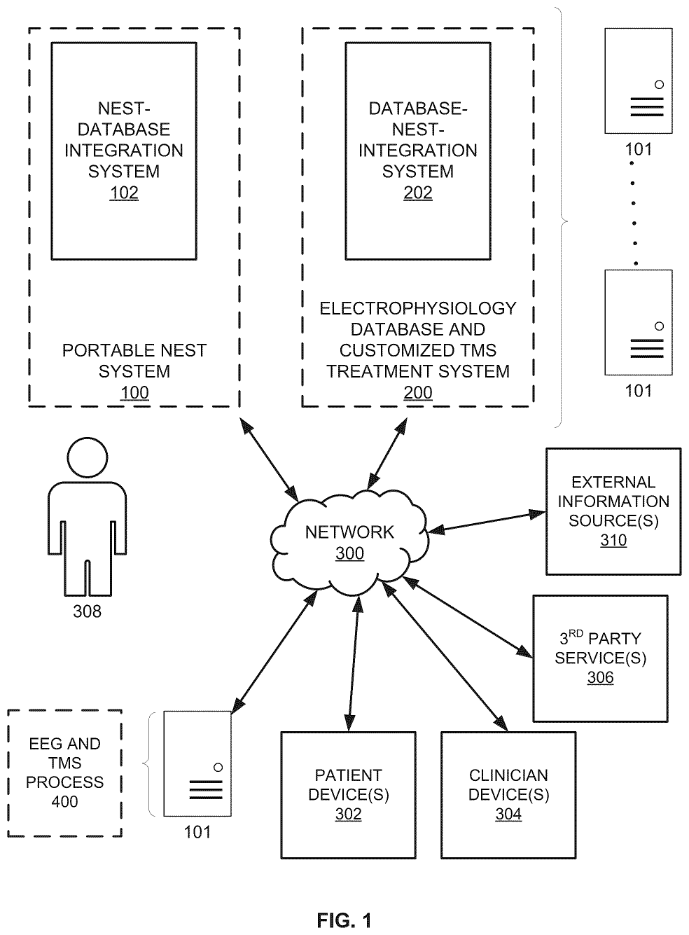

Turning now to FIG. 1 an example environment. The example environment includes a portable NEST system 100 with a NEST database (e.g., NEST-database integration system 102) in communication with a network 300. The network 300 is also in communication with an electrophysiology database and customized TMS treatment system 200 which includes a database integration system (e.g., Database-NEST integration system 202). One or more servers 101 can be provided in communication with the network 300 and/or components in communication with the network 300. Additional components in communication with the network 300 include, for example, external information source(s) 310, third party service(s) 306, clinician device(s) 304, and patient device(s) 302. An EEG and TMS process 400 may run across any combination of the systems and devices. The EEG and TMS process 400 may be run across any combination of an EEG system (such as a portable NEST system 100), an electrophysiology database and customized TMS treatment system 200, and/or another computing device (e.g., one or more servers 101 shown in FIG. 1). The EEG and TMS process 400 may be executed and run on the various systems and computing devices via a network 300. In FIG. 1, the portable NEST system 100 may communicate with the electrophysiology database and customized TMS treatment system 200 according to example embodiments of the disclosure. The portable NEST system 100 may use a NEST-database integration system 102 to communicate and interact with the electrophysiology database and customized TMS treatment system 200 via the network 300. Similarly, the electrophysiology database and customized TMS treatment system 200 may use a database integration system (e.g., Database-NEST integration system 202) to communicate and interact with the portable NEST system 100 via the network 300. Other devices (e.g., various computing devices or mobile devices) such as patient device(s) 302, clinician device(s) 304, and third-party service(s) 306 may communicate with the electrophysiology database and customized TMS treatment system 200 and the portable NEST system 100. The electrophysiology database and customized TMS treatment system 200 and the portable NEST system 100 may also communicate and interact with external information source(s) 310 via the network 300. As shown in FIG. 1, the portable NEST system 100 may be positioned on the head of a patient 308.

The portable NEST system 100 may capture EEG data from a patient 308. The electrophysiology database and customized TMS treatment system 200 may receive the EEG data from the portable NEST system 100. The electrophysiology database and customized TMS treatment system 200 may determine a TMS treatment based on the received EEG data. The portable NEST system 100 may provide sTMS based on the determined TMS treatment. The electrophysiology database and customized TMS treatment system 200 may also access the EEG data for a patient 308 and may classify the patient 308 as having a particular brain type based on the EEG data. The electrophysiology database and customized TMS treatment system 200 may determine a TMS treatment based on the classified brain type and the EEG data. The electrophysiology database and customized TMS treatment system 200 may recommend synchronized transcranial magnetic stimulation (sTMS) based on the determined TMS treatment. In some examples, the portable NEST system 100 may capture the EEG data from the patient 308 that may be accessed by the electrophysiology database and customized TMS treatment system 200. The portable NEST system 100 may provide the recommended synchronized transcranial magnetic stimulation to the patient 308. The electrophysiology database and customized TMS treatment system 200 may access the EEG data for a patient 308 and may analyze the EEG data to indicate brain health of the patient 308. The electrophysiology database and customized TMS treatment system 200 may determine TMS treatment based on the brain health indication and the EEG data. The electrophysiology database and customized TMS treatment system 200 may recommend sTMS based on the determined TMS treatment. In some examples, the electrophysiology database and customized TMS treatment system 200 may provide reporting of the brain health indication in the form of a brain health indicator for the patient 308. The electrophysiology database and customized TMS treatment system 200 may also use the brain health indication for diagnosing the patient 308. The electrophysiology database and customized TMS treatment system 200 may access the EEG data for a patient 308 and may provide bursting analysis of the EEG data. The electrophysiology database and customized TMS treatment system 200 may determine a TMS treatment based on the bursting analysis and the EEG data. The electrophysiology database and customized TMS treatment system 200 may also recommend sTMS based on the determined TMS treatment. Bursting analysis may be used for diagnosing the patient 308. The bursting analysis may be used for determining treatment parameters. The portable NEST system 100 may also capture EEG data from a patient 308 and have one or more rotating magnets for generating an alternating magnetic field. The portable NEST system 100 may determine a TMS treatment recommendation based on the EEG data or may receive the TMS treatment recommendation from a second system based on the EEG data. The portable NEST system 100 may provide sTMS by using the rotating magnets to generate the alternating magnetic field based on the determined TMS treatment or the recommended TMS treatment. The second system may be the electrophysiology database and customized TMS treatment system 200 for receiving the EEG data from the portable NEST system. The electrophysiology database and customized TMS treatment system 200 may determine the TMS treatment based on the received EEG data.

NEST System Components

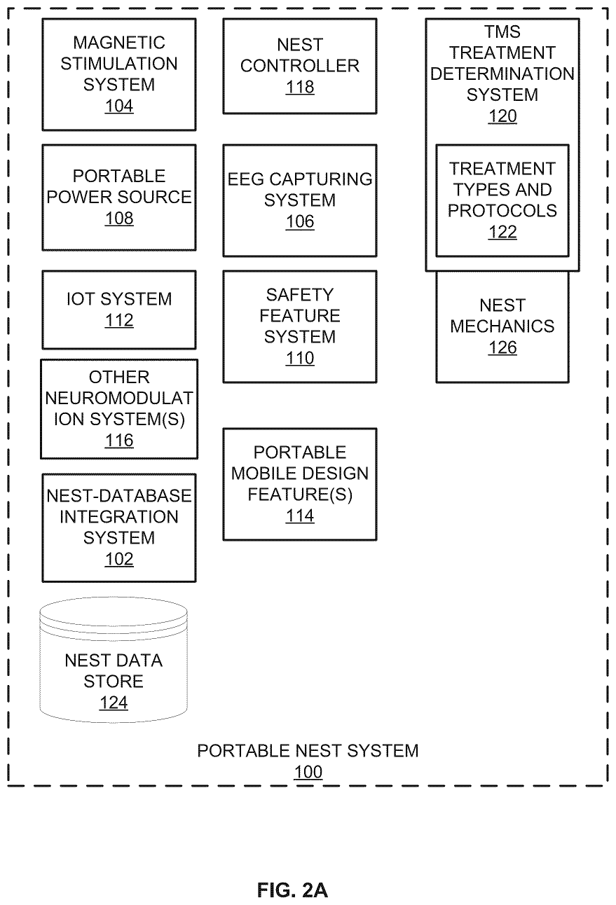

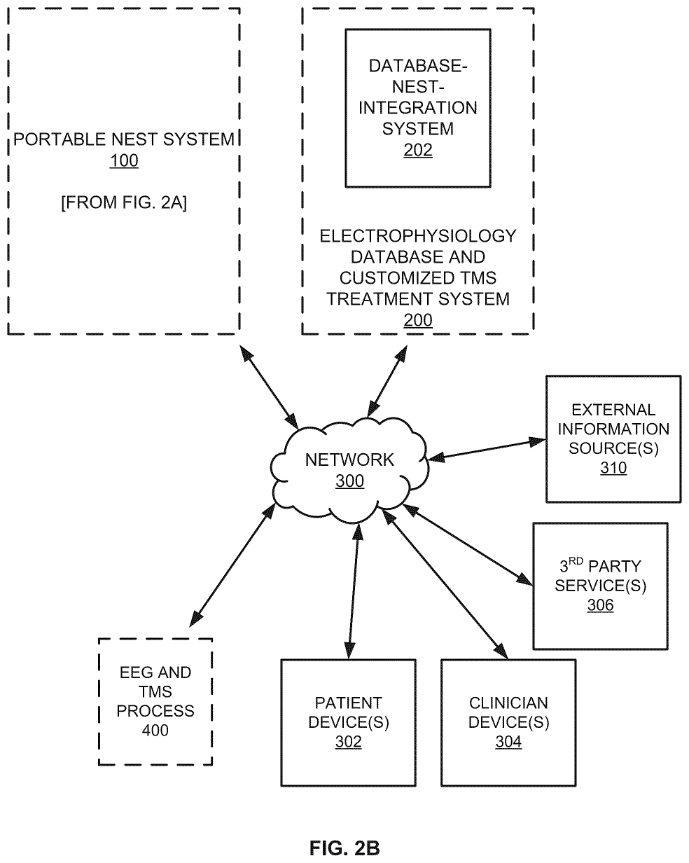

FIGS. 2A and 2B show a detailed view of the portable NEST system 100. The portable NEST system 100 may include the NEST-Database integration system 102 (e.g., portable NEST system software that enables integration with software of the electrophysiology database and customized TMS treatment system 200), a magnetic stimulation system 104 (e.g., including one or more magnets that may provide sTMS), an EEG capturing system 106 (e.g., detecting and capturing EEG data from the patient 308 using sensors such as electrodes placed along scalp of the patient 308), a portable power source 108 (e.g., portable-type power source such as a battery), a safety feature system 110 (e.g., one or more safety mechanisms that may prevent or avoid misuse of the portable NEST system 100 such as a locking mechanism), an Internet of Things (IoT) system 112 (e.g., including various components that may provide IoT capabilities such as relating to connecting the portable NEST system 100 to the network 300), portable/mobile design feature(s) 114 (e.g., one or more features that enable for portability and mobility use of the portable NEST system 100 such as miniaturizing of some features or components), other neuromodulation system(s) 116 (e.g., systems that provide other therapies such as light stimulation therapies), a NEST controller 118 (e.g., controller may be used for managing and directing one or more components of the portable NEST system 100), a TMS treatment determination system 120 (e.g., system for determining repetitive TMS (rTMS) and sTMS that may be based on the EEG data obtained by the EEG capturing system 106) having treatment types and protocols 122 (e.g., information and/or data related to treatment types and protocols as described in the disclosure that may be used by the TMS treatment determination system 120), a NEST data store 124 (e.g., storing any and all data related to use of the portable NEST system 100 and possibly some data received from other systems and/or devices such as received from the electrophysiology database and customized TMS treatment system 200), and NEST mechanics 126 (e.g., various mechanical parts relevant to configuration of the portable NEST device, such as an example portable headset device, for use by the patient 308). The network 300 can be connected to one or more patient device(s) 302, one or more clinician device(s) 304, third party service(s) 306, and external information source(s) 310. The headset device provides a support framework with attached electronic devices that is worn on the head of the user. The form factor of the headset can be a cap, helmet, or hat.

IoT Capabilities

The portable NEST system 100 may be a portable unit device that may provide sTMS where the portable unit device may have internet of things (IoT) capabilities from being connected or when connected to a network (e.g., network 300). The portable NEST system 100 may include the IoT system 112 for providing the IoT capabilities. The portable NEST system 100 may include a small portable TMS type device that may be utilized by patients for home use such that this portable NEST system 100 may be connected as an IoT device to the network 300. Such devices may require strict and effective safety features to prevent misuse (intentional or otherwise). The IoT system 112 may provide an interface via Bluetooth® technologies to a mobile device or a cell phone for the portable NEST system 100. Each person that uses the portable NEST system 100 may have a recorded EEG (e.g., received by the EEG capturing system 106) such that the recorded EEG data may be stored in a cloud, and the recorded EEG or stored variables relating to the recorded EEG may be accessed by the portable NEST system 100 via a Bluetooth® connection for storing EEG data or variables relating to the recorded EEG into the cloud directly. The portable NEST system 100 (e.g., handheld device) may obtain treatment parameters from the cloud. In examples, the EEG data may be reported such that the data may be sent up to a cloud database, there may be an automated algorithm that may evaluate the EEG data for determining a brain's intrinsic frequency. From this information, treatment parameters may be generated and downloaded to the portable NEST system 100 (e.g., handheld device).

Other uses of the portable NEST system 100 as an IoT device are also contemplated. For example, connecting the portable NEST system 100 as an IoT device to a network 300, allows the NEST controller 118 to interface via Bluetooth® to a cell phone. The IoT system 112 of the portable NEST system 100 may include a handheld module or laptop PC or desktop PC that may run an application such that the application may be responsible for setting the treatment parameters in the control module and may also be responsible for setting an available number of treatments for billing purposes and other similar issues. The portable NEST system 100 may generate a database of EEGs (e.g., using the EEG capturing system 106) that are stored locally or in the cloud (e.g., EEG data may be stored initially and temporarily in the NEST data store 124 which may be transferred to a cloud database) that may be available for research and for tailoring an automated algorithm. Developers may be able to view and monitor the accuracy of the automated algorithm. Every patient may have an account and every account may have a number of sessions or amount of time, and treatment parameters. Additionally, every treatment may be logged so that clinicians may go back and view log files to make sure that patients at home may be using the portable NEST system 100 appropriately. This may be fairly unique in that many times, for example, with a medication, the physician may have no idea that the medication is actually being taken on time. This may provide real-time feedback to the clinician that the patient may be complying or not complying with treatment.

Safety Features to Prevent Misuse

The portable NEST system 100 may have the safety feature system 100 that includes safety features for preventing misuse of a TMS therapy (e.g., preventing misuse of the portable NEST system 100). In general, the safety feature system 110 may prevent misuse of TMS with respect to safety limitations. TMS-type devices may require strict and effective safety features to prevent misuse (intentional or otherwise). In some examples, trials and studies may have been about thirty minute sessions with stimulation for about sixty seconds or every minute. One of the safety limitations of TMS may be that each patient may only stimulate for about five to six seconds per minute where there may be thirty cycles for the thirty-minute session. This is to prevent overheating of brain tissue with extended stimulation, which may cause some significant damage if stimulation is applied for too long. The safety feature system 110 of the portable NEST system 100 may limit stimulation to relatively low level of energy (e.g., less than about one percent of what a typical TMS device provides). This safety feature may prevent overheating of the brain tissue such that the device could be used for hours, days, or indefinitely.

The portable NEST system 100 may allow for treatment about once a day. The safety feature system 110 may provide the ability to restrict treatment to be only allowed for thirty minutes per day and each patient user may have their own unique ID. The safety feature system 110 may have a phone or mobile application that may not allow patients to use the portable NEST system 100 more than once per day (e.g., user ID may be restricted for the user ID associated with patient on the phone application). The magnets themselves may have a natural maximum energy stimulation that they can provide such that over stimulation may be avoided. In addition, the stimulation from the magnets may be weak enough such that the patient could use the portable NEST system 100 (even without the safety restrictions) for longer periods of time without any safety risk. The safety feature system 110 may provide a TMS-related work threshold test where the portable NEST system 100 may be positioned over the patient's motor cortex and pulses may be generated looking for whether the patient's thumb twitches. Then, the safety feature system 110 and the TMS treatment determination system 120 may be used to adjust the amplitude based on the results of the TMS-related work threshold test. This may be an example of the energy level that may be set for the patient (e.g., with respect to the patient user ID) when using the portable NEST system 100. In general, the portable NEST system 100 may be considered safer than most conventional TMS devices in that the portable NEST system 100 may avoid dealing with conventional TMS device-type problems such as overcharging of a capacitor causing the capacitor to explode or fail, too much current being sent, and overheating coil that may cause the coil to melt or fail to function as required. Further, the portable NEST system 100 may use the safety feature system 110 to provide additional safety precautions as described in the disclosure.

The portable NEST system 100 (e.g., in the form of a portable unit device) may include the safety feature system 110 having safety features that may prevent misuse of TMS therapy for the device. There are various examples of these safety features as described in this disclosure. Another example safety feature may be tamper proofing (e.g., tamper resistance) for the portable NEST system 100.

Electrophysiology Database and Customized TMS Treatment System—Components

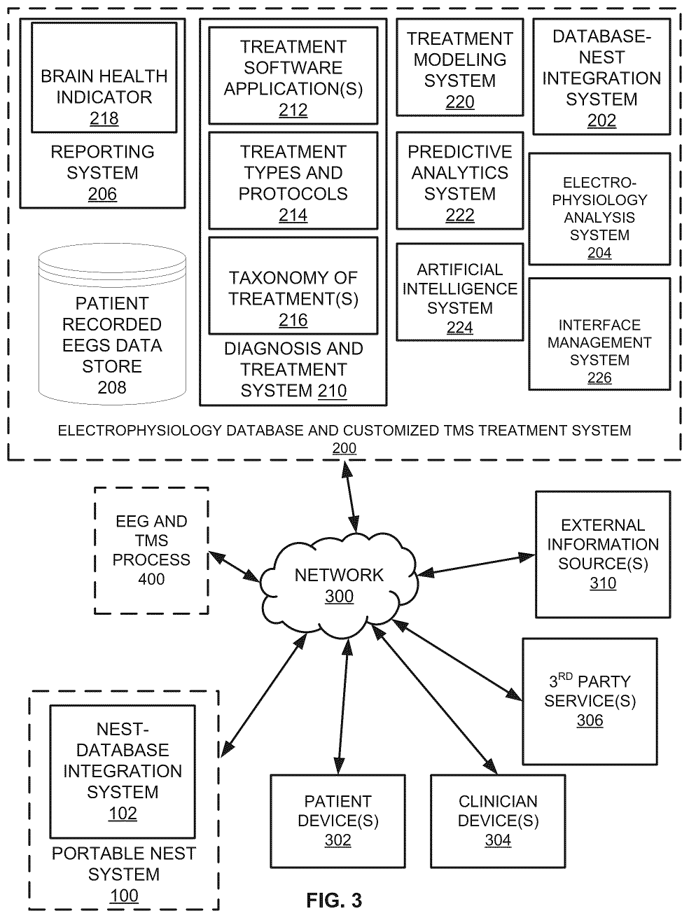

Referring now to an example implementation, FIG. 3 shows a detailed view of the electrophysiology database and customized TMS treatment system 200 in the example environment is shown according to one or more example embodiments of the disclosure. The electrophysiology database and customized TMS treatment system 200 may include the database integration system (e.g., database-NEST integration system 202, or a database software that may enable integration with software of the portable NEST system 100 via the NEST-database integration system 102), an electrophysiology analysis system 204 (e.g., providing various types of analysis of EEG data and other relevant information), a reporting system 206 (e.g., providing various types of reporting such as diagnosis, brain health, treatment recommendations, etc. based on EEG data and EEG data analysis) having a brain health indicator 218 (e.g., showing brain health status based on EEG data), a patient recorded EEGs data store 208 (e.g., data store including pre and post treatment EEG recordings and other EEG-related data), and a diagnosis and treatment system 210 that includes treatment software application(s) 212 (e.g., software applications that may be used by clinician users and patient users with respect to diagnosis and treatment of the patient), treatment types and protocols 214 (e.g., information and/or data related to treatment types and protocols as described in the disclosure that may be used by the diagnosis and treatment system 210), and a taxonomy of treatment(s) 216 (e.g., taxonomy of treatment types and equipment types). The electrophysiology database and customized TMS treatment system 200 may also include a treatment modelling system 220 (e.g., uses big data analytics and/or aggregated data analytics to provide modelling of various treatments in terms of results and impacts), a predictive analytics system 222 (e.g., provide predictive analytics based on big data analytics and/or aggregated data analytics used to identify patients and provide an appropriate treatment for the patients based on EEG data), an artificial intelligence system 224 (e.g., using machine-learning of the artificial intelligence for determining diagnosis and treatment for each patient), and an interface management system 226 (e.g., managing interfaces such as host, patient, clinician, insurance provider, payer, and developer interfaces).

The electrophysiology database and customized TMS treatment system 200 may be software that provides various types of analysis of EEG data. The patient recorded EEGs data store 208 of the electrophysiology database and customized TMS treatment system 200 may have a database of thousands of EEGs (e.g., thirty thousand or more of EEGs). In some examples, the EEGs may be ported into an object storage service (e.g., simple storage service (S3) of Amazon having buckets such as file folders and store objects that may include data and descriptive metadata) which may be in a data store or database where the EEG data may be analyzed.

The electrophysiology database and customized TMS treatment system 200 may include interfaces (e.g., controlled by the interface management system 226) that may be built in the dashboards (e.g., data dashboards).

Electrophysiology Database and Customized TMS Treatment System—Platform

The electrophysiology database and customized TMS treatment system 200 may be a customized TMS treatment platform. The customized treatment platform may provide TMS treatments based on each patient. As described in the disclosure, TMS may be a non-invasive technique, where in some examples; a small coil may be placed over a patient's scalp. The electric current circulating through the coil may produce a magnetic field, which can then pass through the scalp and bone and induce changes in nerve cell activity in the cortex. TMS may improve cognitive health by improving cortical network coherence and brainwave synchrony. This electrophysiology database and customized TMS treatment system 200 may be a platform that may enable patients to engage in the process of healing their brain to help them feel a part of the process. Patients may be more likely to seek continued treatment if they find it to be an enjoyable process.

The customized TMS treatment platform may provide TMS treatment that may be a personalized TMS directed to where a frequency of magnetic pulses may be determined based on a patient frequency. The provided TMS treatment based on frequency may be a recommended treatment that may be determined and provided by the diagnosis and treatment system 210. This patient frequency may be an intrinsic alpha frequency that may be a brain resonant phenomenon. This may involve targeting a location along with using other potential technologies. The customized TMS treatment platform may enable EEG data from individuals to be used for determining a frequency that TMS may use to improve mental health. Two emergent forms of TMS may include theta-burst magnetic stimulation (TBS) and EEG-based sTMS. TBS may involve use of a triple-pulse burst in either a continuous or an intermittent form and may be thought to induce rapid effects, while it may be the intent of sTMS to identify the most optimal stimulation protocol for an individual patient in real-time.

The customized TMS treatment platform may provide TMS treatments that may be used to provide improved inhibitory control for ameliorating symptoms in a range of mental disorders. Performance in cognitive processes may be modulated by application of TMS dependent on technical parameters (e.g., intensity, duration, offline/online, polarity, placement of tesla coil, etc.) and task parameters (e.g., task, instruction, stimuli, outcome recordings), which may involve a specific neurocognitive function such as inhibitory control.

The customized TMS treatment platform may include one or more types of interfaces for users. For example, a variety of types of interfaces may be used with the platform in providing treatments to patients. The interface management system 226 may manage and assist with use of these different interfaces. In examples, users may be clinicians and/or patients (may use the platform via the clinician device(s) 304 and/or the patient device(s) 302, respectively). The users (e.g., related to the one or more types of interfaces) may include a host user, a patient user, a clinician user, an insurance provider user, a payer user, and a developer user. The clinician user may be a doctor that may work at a clinic using the customized TMS treatment platform for patients. The insurance provider user may be an insurance company that may cover costs related to use of the TMS treatment platform (e.g., may use 3rd party service(s) 306 to access platform of the electrophysiology database and customized TMS treatment system 200). The payer user may directly cover costs related to the customized TMS treatment for a particular patient. This may be a parent for a child patient or a son/daughter of an elderly patient. The developer user (e.g., software developer) may continuously monitor and update software of the platform as improvements may be needed to the functionality of the platform.

The customized TMS treatment platform may have EEG data that includes EEGs from pre and post treatments (e.g., as stored in the patient recorded EEGs data store 208) for providing feedback in terms of diagnosis and treatment of patients, and the platform may generate reporting based on the EEGs. This reporting may be generated by the reporting system 206. In some examples, the EEGs from pre and post treatments may be EEGs from before and after use of the portable NEST system to indicate a set of effects of the TMS treatment.

Electrophysiology Database and Customized TMS Treatment System—Comparison to Other EEG Databases

As described in the disclosure, there may be normative EEG databases in existence that few universities have created and used. However, when it comes to non-normative data, there do not seem to be EEG databases that provide large autism or PTSD EEG data sets that may be norms for PTSD or for autism. Generally, a clinical score may be an instrument such as a quality-of-life measure. The quality-of-life measure may be obtained from people who have no quality-of-life disruption. The quality-of-life measure may be obtained from people who have a diagnosis of post-traumatic stress disorder. As a comparison for PTSD, quality of life measures may be taken from people who have been deployed versus people who have never been deployed. For all of these different measurements, the EEG and TMS process 400 may use the electrophysiology database and customized TMS treatment system 200 to obtain a normative value. When obtaining new measurements for a patient, the electrophysiology database and customized TMS treatment system 200 may compare the patient's history and/or that patient's diagnosis against what the normative may be in order to see to what extent the normative may vary from the average number for patients by age who have been tested. A standard deviation may be calculated for each patient as well by which the electrophysiology database and customized TMS treatment system 200 may then compare how much higher or lower than normal the patient's score may be for the quality-of-life measure. For EEG data, similar processes may be used for the normative databases that may be receiving EEG data from patients. The electrophysiology database and customized TMS treatment system 200 may analyze each of the bands of activity from slow activities (delta and theta) to faster rhythms (alpha and beta). The EEG and TMS process 400 may use the electrophysiology database and customized TMS treatment system 200 to determine how much more or less of the rhythm, does this person have for their age. This may be determined based on standard deviation. The electrophysiology database and customized TMS treatment system 200 may also determine how much higher or lower the data is relative to the normative database. To classify data as normative, these scales may be needed. In some examples, the electrophysiology database and customized TMS treatment system 200 may include EEG data or EEGs on patients as they receive treatment over time.

The electrophysiology database and customized TMS treatment system 200 may be created from gathering of EEG information and data on patients (e.g., from clinics). The electrophysiology database and customized TMS treatment system 200 may also receive and/or obtain reporting of scales (e.g., scale information) from clinics.

The electrophysiology database and customized TMS treatment system 200 may be built as data stores or databases that may accrue the EEG data (e.g., stored in the patient recorded EEGs data store 208). The EEG data may include EEGs and scales on different diagnostic groups that may be administered and provided by clinicians. The electrophysiology database and customized TMS treatment system 200 may aggregate the EEG data. In some examples, patients may sit in front of a computer screen and run a task and record the EEG for the patient during completion of the task (e.g., task related EEG). The EEG and TMS process 400 may use the electrophysiology database and customized TMS treatment system 200 with the portable NEST system 100 to provide slight sounds and sights that may be related potentials that may be measured in the EEG.

Typically, the normative databases (e.g., at NYU and Maryland) may obtain resting EEG measurements when patient's eyes are closed while awake in some examples. There may be sleep EEG normative databases. The electrophysiology database and customized TMS treatment system 200 may obtain EEG measurements for patients when their eyes may be closed (e.g., may be referred to as resting awake EEGs). The electrophysiology database and customized TMS treatment system 200 may obtain eyes open resting EEGs and may obtain task-related EEG. There are advantages to having patients sit down and having the patient's EEG measured for ten minutes. Then, the electrophysiology database and customized TMS treatment system 200 may aggregate that data. In some examples, EEG data may be obtained or taken in through structured query language (SQL) and may be a hosted SQL database with no real organization. Data dashboards may be included and used with respect to analysis of EEGs (e.g., EEG data).

Electrophysiology Database and Customized TMS Treatment System in General

When the EEG and TMS process 400 may use the electrophysiology database and customized TMS treatment system 200 to analyze EEG data when the patient may close their eyes such that it may be expected that main activities may be associated with frequency in the range of about 8 to about 13 Hz alpha range which is where most of the rhythm may occur. The sampling rates for EEG may be about one hundred Hertz (or about one-hundred times per second, about two-hundred times per second, about five-hundred times per second, etc.). Then, there may be information at half a sampling frequency essentially in terms of what it may be that the electrophysiology database and customized TMS treatment system 200 may analyze as captured by the portable NEST system 100 (e.g., captured EEG activity or neuronal firing rates). Brain cells may typically be communicating with each other but may vary this communication. For example, during a ten minute EEG with eyes closed, the electrophysiology database and customized TMS treatment system 200 may detect frequency activity or content in delta at about 1 to about 4 Hz, in theta at about 4 to about 8 Hz, in alpha at about 8 to about 13 Hz, in beta at about 13 to about 30 Hz, in gamma at about 30 to about 40+Hz. All of these different frequencies may interact with one another. When it comes to measuring these frequencies, the traditional approach may be to look at the wide band. The electrophysiology database and customized TMS treatment system 200 may monitor and analyze delta, theta, alpha, and/or beta which may be wide frequency ranges and look at, for example, power under a curve or all content as a measurement. The EEG and TMS process 400 may use the electrophysiology database and customized TMS treatment system 200 initially then may measure these frequencies by each individual electrode comparison to other electrodes. For example, if there are nineteen electrodes on the portable NEST system 100 (e.g., on the head cap device), there may be a 19×19 measurement matrix where every measurement for each of these wide bands may be a comparison between single electrodes. The following issues may be considered: cluster electrodes may add complexity and looking at one hemisphere versus another hemisphere may be another layer of complexity. A single measurement may be to look at power (e.g., an amplitude of an activity) under an electrode, then have a couple hundred measurements may be immediately determined from monitoring of the wide band.

When it comes to the way that brain activity may be coming about in a patient's cortex, the brain activity may come in narrow frequency bursts (e.g., waves that may be repeated). These waves may be repeated without too much frequency variation. For example, one patient may have a burst of theta activity at about 6 or about 6.3 Hz that may come about every now and then in EEG data. The EEG and TMS process 400 may use the portable NEST system 100 to measure most (if not all) of theta and may store this data in the electrophysiology database and customized TMS treatment system 200. In some examples, the electrophysiology database and customized TMS treatment system 200 may measure more narrowly at about 6 to about 7 Hz. The EEG and TMS process 400 may use the portable NEST system 100 to capture about 6.3 Hz (which should be within about 6 to about 7 Hz). When it comes down to analysis, the electrophysiology database and customized TMS treatment system 200 may use patient's frequency to find frequency bins (e.g., one-Hertz bins). In examples, the electrophysiology database and customized TMS treatment system 200 may slice zero up to about forty in one Hertz bins such that the electrophysiology database and customized TMS treatment system 200 may have 40 bins times a matrix that matches the dimensions of electrodes used in recording (e.g., a 19 by 19 matrix when there are 19 EEG data sources), or EEG measurement alone and the numbers may continue to increase. This n-by-n matrix of data with 40 or more or less frequency bins may apply to each measurement of data.

The electrophysiology database and customized TMS treatment system 200 may analyze EEG coherence. This may be a zero to one measure of mutual information, and how related the electrodes are to one another which may be another 19 by 19 matrix by the number of frequency ranges being viewed. The electrophysiology database and customized TMS treatment system 200 may analyze with respect to phase lag or bursting metrics or other metrics. Thus, the electrophysiology database and customized TMS treatment system 200 may provide for a relatively rich numeric database that may have a relatively large amount of EEG data (e.g., in the patient recorded EEGs data store 208). The electrophysiology database and customized TMS treatment system 200 may use the artificial intelligence system 224 to provide machine learning such that the artificial intelligence system 224 may use the data in order to have input by which the artificial intelligence system 224 may specify some output and learn from the output. A statistical analysis plan may be used in setting up one or more portions or components of the electrophysiology database and customized TMS treatment system 200. The electrophysiology database and customized TMS treatment system 200 may have a goal of circulating around determining ideal frequencies for treatment which may be based on a clustering of resting activity references (e.g., eyes closed EEGs). The patient recorded EEGs data store 208 may include any number of recorded EEGs and related EEG data (as may only be restricted by size of computing storage). The patient recorded EEGs data store 208 may include 30,000 scans that may be increasing continuously. The electrophysiology database and customized TMS treatment system 200 may use patient recorded EEGs data store 208 for diagnosing PTSD, autism spectrum disorder (ASD), development delay, etc. for patients. The electrophysiology database and customized TMS treatment system 200 may use the clustering of different densities of delta, theta, alpha, beta, in terms of amplitude and other measurements (from EEG data or other data) for providing diagnoses (e.g., including understanding of variation in diagnoses) and treatment parameters (e.g., TMS treatments). Specifically, the electrophysiology database and customized TMS treatment system 200 may use this EEG in understanding variation in diagnoses. The electrophysiology database and customized TMS treatment system 200 is also unique in that the patient recorded EEGs data store 208 may include pre and post treatment EEGs related to TMS.

The patient recorded EEGs data store 208 may include EEG data and information as baseline and then have EEG data and information after patients have received treatment (specifically TMS treatment) such that electrophysiology database and customized TMS treatment system 200 (e.g., using the electrophysiology analysis system 204) may monitor for how clustering may shift or change in response to therapy by the recorded clinical skills that may also be shifting. The electrophysiology database and customized TMS treatment system 200 may specifically be looking for trends in cluster adjustment. If the electrophysiology database and customized TMS treatment system 200 may set lower and upper bounds for these calculations, the electrophysiology database and customized TMS treatment system 200 may be able to have a better understanding when a patient indicates that they have ASD (e.g., selects ASD box when filling out clinical form) where by all measurements that may have been gathered by the EEGs and the clustering that may have been determined may indicate where the patient fits or falls in a large cluster of metrics (e.g., clusters). The electrophysiology database and customized TMS treatment system 200 may be used to determine how severe the patient's brain may be compared to other “normal” patients or other patients with a similar diagnosis in the same age range, same sex, same race, etc. as the patient. The electrophysiology database and customized TMS treatment system 200 may also provide a diagnosis for the patient as well as determine where the patient may fit in terms of their brain health compared to other normal patients and/or patients having similar diagnoses (e.g., providing reliability analysis by using the electrophysiology analysis system 204 to determine a reliability of a diagnosis based on data) and/or may be used in combination with third party normative databases (e.g., may use 3rd party service(s) 306 to access the electrophysiology database and customized TMS treatment system 200) for comparison in terms of diagnosis (e.g., against normative patients having similar or the same diagnosis) to improve quality of diagnosis by the electrophysiology database and customized TMS treatment system 200.

The electrophysiology database and customized TMS treatment system 200 may provide a database with a variety of advantages and benefits as compared to other EEG databases (e.g., normative databases). The electrophysiology database and customized TMS treatment system 200 may be configured to provide a unique relatively large group of pre and post treatment recorded EEGs and EEG data that may increase over time and may be extremely versatile and useful for various applications.

Electrophysiology Database and Customized TMS Treatment System—Patient Recorded EEGs Data Store

The electrophysiology database and customized TMS treatment system 200 may include the patient recorded EEGs data store 208 that may be an electrophysiology database or database system having electroencephalograms (EEGs) from pre and post treatments that may provide feedback in terms of diagnosis and treatment of patients. The patient recorded EEGs data store 208 may be configured to provide a unique relatively large group of pre and post treatment recorded EEGs and EEG data that may increase over time and may be extremely versatile and useful for various applications (e.g., applications in terms of artificial intelligence and machine learning). The EEG data (e.g., recorded EEGs) in the patient recorded EEGs data store 208 may be analyzed for determining diagnostics and treatments for patients (e.g., may be based on mental disorders).

The patient recorded EEGs data store 208 may be formed and updated with new EEG data by a process (e.g., the EEG and TMS process 400). In one example, the EEG and TMS process 400 may include recorded EEG data that may be placed in an SQL database or data lake or lake house (e.g., of the patient recorded EEGs data store 208). A patient may come into a clinic where they may be seen by a clinician. The patient may have their EEG taken while they sit down with their eyes closed for about ten minutes. The clinic may use the EEG and TMS process 400 to share and transfer the patient's EEG data with the electrophysiology database and customized TMS treatment system 200. This EEG data may be transferred to the electrophysiology database and customized TMS treatment system 200 (e.g., to and/or from servers of the system 200). Associated with this EEG data may be data that may include information about the patient such as some clinical information or scales in addition to EEG data. The electrophysiology database and customized TMS treatment system 200 may provide back some frequency settings for TMS treatment (e.g., to the portable NEST system 100). The electrophysiology database and customized TMS treatment system 200 may hold for about two weeks when the electrophysiology database and customized TMS treatment system 200 receives another new set of EEG data after two weeks of TMS treatment (e.g., by the portable NEST system 100) in addition to an update on what may be happening with the patient and then this update may occur every two weeks for as long as the patient may continue to receive treatment therapy (e.g., TMS treatment from the portable NEST system 100). The average course of therapy or treatment may be about six weeks. A majority of patients may be coming in for two-week periods to be treated and/or monitored by recording EEGs and determine if the patient may feel better and/or improvements in behaviors (e.g., kids with mental disorders may be talking or may be providing more eye contact). After two-week period, some patients may enter into a whole month of therapy where EEGs may be provided to electrophysiology database and customized TMS treatment system 200 at four weeks and at six weeks from the baseline. Most patients may have about three or four EEG recordings and if the patient may continue for another month, the patient may have additional EEG recordings. This may be the process used to collect EEG recordings for the patient such that the electrophysiology database and customized TMS treatment system 200 may receive this EEG data and may provide back analysis on the EEG data to clinicians and/or patients regarding diagnoses and therapy settings for treatments (e.g., settings for the portable NEST system 100 regarding TMS treatments). The EEG data may be placed in the object storage service (e.g., S3 bucket of Amazon as described in the disclosure) of the patient recorded EEGs data store 208 such that the EEG data may be generally in the same place and may be labeled. This EEG data in the patient recorded EEGs data store 208 may be easily searchable, queried, and clustered. The EEG data may be used for diagnosis and for identifying possible improvements or changes of symptoms or changes of EEGs because of treatments.

The electrophysiology database and customized TMS treatment system 200 may include the patient recorded EEGs data store 208 that may be an electrophysiology database or database system having electroencephalograms (EEGs) from pre-treatment and post-treatment that may provide feedback in terms of diagnosis and treatment of patients such that the database or database system of the patient recorded EEGs data store 208 may include an object storage database (e.g., object storage service) that may store data from the EEGs as objects (e.g., a set of objects). For example, EEG data may be stored into the SQL database (e.g., recorded EEG data that may be placed in an SQL database of the patient recorded EEGs data store 208). Data from EEGs may be received by the patient recorded EEGs data store 208 (e.g., including system servers) along with information portions about the patient that may be tied with the EEG data (e.g., labels and/or directly or indirectly linked) relating to each patient. These information portions may include chief complaints and other clinical information or scales from clinics. The electrophysiology database and customized TMS treatment system 200 (specifically the patient recorded EEGs data store 208) may provide a proper software environment or proper database for the EEGs where the data may be easily queried and explored (e.g., S3 buckets as described in the disclosure).

Electrophysiology Database and Customized TMS Treatment System—Treatment Software Application

The electrophysiology database and customized TMS treatment system 200 may include the diagnosis and treatment system 210 having treatment software application(s) 212 for providing a customized treatment software application. The electrophysiology database and customized TMS treatment system 200 may include the customized treatment software application for providing TMS determined treatments based on EEG data. The customized treatment software application may provide TMS suggested treatments to a clinician as determined for each patient such that the software application may be configured to execute these suggested treatments through engagement with each patient. The electrophysiology database and customized TMS treatment system 200 (e.g., platform type system) may also allow patients to generally engage in the process of healing their brain by using an application on their patient device(s) 302 (e.g., mobile device, phone, tablet, or other computing devices). This platform system may allow patients to engage in the process of healing their brain with a device (e.g., portable NEST system 100) with the patient device(s) 302 and find the process enjoyable.

The customized treatment software application may determine TMS treatments based on a gamification application. Gamification of the TMS treatment may use the electrophysiology database and customized TMS treatment system 200 (e.g., platform-type system) that may allow for TMS treatment to be gamified, potentially by showing patients how they can control their own brain and giving them a limited ability to observe how it works for themselves. Safety locks may be used to prevent misuse where a trained professional may not be present to manage the TMS treatment. Such a customized TMS treatment system may include an EEG system as well as may be used as part of neurofeedback, where the patient may take an active part in controlling their brainwave activity along with stimulation from the TMS treatment system (e.g., electrophysiology database and customized TMS treatment system 200).

Electrophysiology Database and Customized TMS Treatment System—Treatment Type Taxonomy

The electrophysiology database and customized TMS treatment system 200 may include the diagnosis and treatment system 210 having the taxonomy of treatment(s) 216 for providing TMS treatments selected from a taxonomy of treatments. The taxonomy of treatment(s) 216 may be part of a platform that may allow for categorization of treatment types into a hierarchal taxonomy. This hierarchical taxonomy may allow for summarization of specific treatment types, while allowing for differentiation of results as finer grained treatments may be conducted. The categorization may include factors such as positioning of stimulation components (e.g., positioning of coil or magnets), magnetic field characteristics, output waveform, magnetic field spatial distribution, magnetic field strength gradient, device compatibility, and safety features. The taxonomy of treatments may be categorized by TMS treatment types and/or TMS equipment types.

Electrophysiology Database and Customized TMS Treatment System—Reporting System (Brain Health Indicator or Brain Synchrony Indicator)

The electrophysiology database and customized TMS treatment system 200 may include the reporting system 206 having the brain health indicator 218 (e.g., provides a report of the brain health indicator 218 based on EEG data). The electrophysiology database and customized TMS treatment system 200 may also provide diagnosis based on the brain health indicator 218. The brain health indicator 218 may relate to a relative brain synchrony index (RBSI). The brain health indicator 218 may also provide a number value that may provide an indication of a patient's brain health or brain state. In some examples, the reporting system 206 may generate a report that may include the brain health indicator 218 in different forms that may be based on different processes. In some examples, the electrophysiology database and customized TMS treatment system 200 may provide a report including or providing a diagnosis and a recommended determined treatment based on EEG data.

As background, when young between about ages two and four, there may be physiological development of cortical tissue as people grow that may accommodate for a relative fast resting frequency. For example, at a younger age (e.g., about 1.5 years old or younger), EEG activity for most normal patients may be about 4 to 6 Hz as a baseline when eyes may be closed. Between the ages of about 1.5 to about 2 years old, the resting eyes close frequency may develop at and above about eight Hertz for the EEG. This function may be in the alpha range (e.g., alpha wave or brain clock) which may be responsible for the speed at which people may process information cortically locally and globally. As a comparison, considering video in number of frames per second, if the video may be below thirty frames per second (FPS), the view may appear choppy and if the video may be above thirty FPS, the video may appear smooth which may be similar to processing frequency of patients maxing out at about 12 Hz or about half 30 Hz. Frequency may be important to the brain health indicator 218. For example, normally developing children patients may have about 10 to about 11 Hz frequency EEG. With development delayed children patients, the frequency may not increase above eight Hertz. With autistic patients, the EEG may max out at about nine Hertz but there may be a delay in the speed of development. For those patients that are normally developing, they may have a peak frequency at about 11 Hz or about 12 Hz and there may be an average frequency of about 10.5 to 11 Hz for patients that may be particularly healthy in terms of development. For people with no head injuries and/or no substance abuse history or sleep disruption, these frequencies may be stable into mid-thirties or early thirties for people. Then, every decade or so, these frequencies may slow down by about a 0.5 to 0.25 Hz normally. For most (if not all) patients, the electrophysiology database and customized TMS treatment system 200 may identify frequency or speed disruptions such as noticing activity may be slower than what may be expected for patients. One of the inputs of this speed number may be relative speed versus what may be expected to be detected for each patient. With treatment, the electrophysiology database and customized TMS treatment system 200 may direct ratcheting speed up for patients who may be responsive. For example, if the patient may be about nine Hertz, there may be a goal of shifting frequency towards about eleven Hertz. With treatment over a course of a few weeks, the frequency of the patient may increase to about 9.5 or about 9.7 Hz. With another few weeks or months of treatment, the frequency of the patient may ramp up to about 10.5 or about 11 Hz, which may be very positively correlated with the patient's clinical and cognitive output and function.

For perspective, the electrophysiology database and customized TMS treatment system 200 in combination with the portable NEST system 100 (as part of the EEG and TMS process 400) may treat brain activity. For example, EEG data may have a label noting PTSD or traumatic brain injury for a patient. The electrophysiology database and customized TMS treatment system 200 may use a diagnosis specific algorithm built (e.g., a PTSD algorithm) for diagnosing the patients based on EEG data. Several factors may disrupt speed of the patient's brain that may be reinforced by slower activities that may correspond with the slower brain speed. The electrophysiology database and customized TMS treatment system 200 may determine how disrupted the patient's brain may be from the patient's ideal speed (e.g., as calculated for each patient) and a degree of displacement for the patient's relative brain synchrony index or RBSI (e.g., where brain synchrony may refer to how synchronous or how stable the patient's brain function may be at a slower frequency versus the patient's ideal brain). This may provide a relative indication (e.g., in the brain health indicator 218 of the reporting system 206) such as about 70% or about 40% of speed that the patient may be as an indicator for people or patients who may not know much about brainwaves but can measure their EEG and obtain this feedback from the electrophysiology database and customized TMS treatment system 200. For example, a patient may receive a report (e.g., from the reporting system 206) that the patient may be about 80% which may be acceptable to them or the patient may be about 90% with treatment such that the patient may feel better. The electrophysiology database and customized TMS treatment system 200 may use feedback from frequencies or analysis from EEG data that may be compared against other EEG data with similar clusters of symptoms and may feed that EEG data into a larger group of EEG data to calculate on average how those numbers may compare. In addition, the electrophysiology database and customized TMS treatment system 200 may analyze after treatment how much these numbers may have shifted. The reporting system 206 may generate different reporting of the brain health indicator 218 that may show the brain health as part of reports that may include other data, analysis, diagnoses, treatment, etc.

The patient may record a baseline EEG (e.g., from the EEG capturing system 106 of the portable NEST system 100). The EEG may be received by the electrophysiology database and customized TMS treatment system 200, which may use the reporting system 206 to provide a report that may have outputs and analysis. The electrophysiology database and customized TMS treatment system 200 may run any variety of analysis (e.g., using the diagnosis and treatment system 210) from the EEG data and/or treatment information (e.g., as part of the EEG and TMS process). The electrophysiology database and customized TMS treatment system 200 may provide this analysis in the form of reports back to the clinic (e.g., using the reporting system 206). Then, the patient may come to the clinic during the week to get a daily treatment (e.g., magnetic e-Resonance therapy or guided TMS). The magnetic e-Resonance therapy may be referred to as MeRTSM, which may be a treatment that may combine rTMS technologies, quantitative electroencephalogram (QEEG), and electrocardiogram (ECG/EKG) for providing treatments tailored for each patient's brain. Then, the patient may go to the clinic the next week (e.g., Monday through Friday) for daily TMS (e.g., MeRTSM). Then, one week after the initial week the EEG capturing system 106 of the portable NEST system 100 may be used to obtain another EEG from the patient which may be sent to the electrophysiology database and customized TMS treatment system 200. EEGs may be obtained from the patient to monitor whether treatment may be showing successful results and if needed, treatment may be adjusted to improve EEG results, which may be captured again to check whether adjustments affected EEG. Some patients may restart their treatment process again such that EEGs should be captured at restart.

A new EEG may be received after two weeks and a new analysis may be performed that may be reported (e.g., using the reporting system 206) as part of the EEG and TMS process 400. In this report, there may be analyses that the clinician may view and the clinician may view pre, post EEG activity and how the EEG may be different or similar between pre and post EEG activity. The report may show how much the activities of interest impact any shifting or lack of shifting of the EEG data. For example, may be a decrease in an undesired rhythm that may be during waking states and an increase in the rhythm of interest in brain clock activity or a shift in frequency. This reporting may serve as an indication of why it may be important that these changes in frequency may directly correspond with improvements in focus for patients.

Electrophysiology Database and Customized TMS Treatment System—Clustering Analysis

The electrophysiology database and customized TMS treatment system 200 may provide clustering analysis (e.g., using the electrophysiology analysis system 204) that may be used for diagnosis and treatment (e.g., using the diagnosis and treatment system 210). After EEG measurements may be taken, a goal may be set of determining ideal frequency for treatment but may also determine insights from clustering of EEGs or EEG data for one patient against other patients. The clustering may be k-means, KNN, shared nearest network, and the like. For example, as part of clustering process, all of the EEGs may be integrated in one place such that recurring all EEG analysis may be completed and then clustering may be run (e.g., using a preferred K-means).

Electrophysiology Database and Customized TMS Treatment System—Bursting Analysis

The electrophysiology database and customized TMS treatment system 200 may provide bursting analysis (e.g., using the electrophysiology analysis system 204) that may be used for diagnosis and treatment (e.g., using the diagnosis and treatment system 210). Analysis thresholds for identification of specific activities, such as bursts of activities, may be adjusted and normalized to the EEG as EEG information is gathered. In some examples, these bursts may be labeled and noted as objects such that these labeled burst objects may be monitored based on their behavior locally and globally in the brain which may be used as further analysis in tracking progress in response to treatment. When viewing a wave on an EEG trace, the wave may be a representative of an increase and fluctuation in charge density under the electrode. The electrophysiology database and customized TMS treatment system 200 may label bursts of EEG activity in the EEG data of each patient (e.g., labelling bursting or bursts in the EEG data). The electrophysiology database and customized TMS treatment system 200 (as part of the EEG and TMS process 400) may outline use of a continuous wavelet transform (e.g., formal tool that may provide a representation of a signal by letting scale and translation parameters of wavelets vary continuously) in order to identify a specific type of wave in EEG data. In some examples, these techniques may be used to explore and find specific types of waves that may be associated with or may relate to diagnoses (e.g., waves that may have a pattern or signature associated with seizures that may be referred to as “seizure waves”). One example use of continuous wavelet transformation may be an identification of shrinking or increasing of size of waveforms that may be taken through each channel until the electrophysiology database and customized TMS treatment system 200 may identify a representative at least a portion (e.g., wavelets) of the waveform as seizure activity. This representative portion may be referred to as a burst of activity where the tissue itself may essentially be ringing at a specific rate or frequency. In addition to utilizing the continuous wavelet transform to identify wavelets, the electrophysiology database and customized TMS treatment system 200 may also identify where there may be a repetitive solitary activity that may be related two in a row or three in a row. The electrophysiology database and customized TMS treatment system 200 may label bursts that may be noted as objects such that these object-related activities may occur locally and globally in the brain which may be used as another metric to track progress in response to treatment. This metric for burst-related objects and activities may also as be used for another level of analysis and understanding (e.g., as part of the EEG and TMS process 400). For example, other analyses related to bursts may include determination how much power may be in a band, which may be the most basic way of looking at EEG and being able to identify that there may be two hundred bursts wanted when treatment started and there may be two hundred seventy bursts preferred for analysis after a week. This may be a granular analysis where the electrophysiology database and customized TMS treatment system 200 may look for determining the strength of the band that may be moving through cortical tissue, ratio of movement of the ripple band, delay, etc. This may allow for the electrophysiology database and customized TMS treatment system 200 to have another degree of understanding of the health of the bursting activity against other patients who had pre and post treatments from the patient recorded EEGs data store 208 (e.g., database) which may continue to increase with useful data that may be used to provide interesting insights that may relate to bursting. Whenever a new measurement may be discovered, the patient recorded EEGs data store 208 may be analyzed to determine how intervention of particular treatment (e.g., specific TMS treatment parameters) may have impacted EEG data and may have adjusted the EEG data such that this particular treatment may be modeled for use by other patients who may be new patients with similar diagnoses based on EEG data. These modeled treatments may be used to change and improve metrics of the new patients. This additional granular layer and use of bursting as a measurement may allow for the electrophysiology database and customized TMS treatment system 200 to specify any frequency band of burst such as theta (or for about 7-8 Hertz).

In general, EEG activity in posterior brain regions may be important for awareness and sensory input whereas EEG activity in frontal regions may be important for other reasons such as working memory and higher function cognition. How these parts or regions of the brain (front and back) may communicate with each other may be another layer of importance that may provide interesting data related to local function, local stability of measure, global stability of measure, and why patients who may have local stability in posterior regions versus frontal regions may have differences in therapy response but may also have differences in genetic variability as a cluster. These interesting data points may be utilized by the electrophysiology database and customized TMS treatment system 200 to provide additional analyses through identification of EEG endophenotypes or signatures. In some examples, there may be typically about nine microvolts at a frontal theta where about 6.5 Hz frontal bursts (about 50 of them) may be identified. There may be some patients with about fifty frontal bursts in about 6.5 Hz, which may identify and possibly diagnose patients as having alcoholism, substance abuse, a head injury of a certain type, and the like. The electrophysiology database and customized TMS treatment system 200 may take this additional labeling and measurements versus other important clinical insight for providing diagnoses and treatments (e.g., using the diagnosis and treatment system 210). Machine learning from the artificial intelligence system 224 may be used to provide a diagnosis based on percent confidence, a measurement that may be taken as fitting into a bin or folder related to diagnoses. This may be similar to an approach where a blood test may be used to provide a relative percentage of heritability. EEG data may be used to determine a percentage of confidence that a component or portion of the EEG may have a pattern that may be related to reported symptoms and/or diagnoses. As more and more data may be obtained, the number of insights and accuracy may increase that may help add context to EEG data. The electrophysiology database and customized TMS treatment system 200 may include software that may provide questions for patients. The patients may use a mobile device (e.g., iPad) to input answers to these questions (e.g., in a survey at the clinic) before an EEG may be taken (e.g., where EEG may have labels). The electrophysiology database and customized TMS treatment system 200 may use the input answers along with the EEG data for providing diagnoses, brain health indications, and treatments for patients. In some examples, the survey of questions may be in paper form, which may be provided to patients at the clinic. The electrophysiology database and customized TMS treatment system 200 (as part of the EEG and TMS process 400) may provide the ability to scan the paper and convert the scanned document into data (e.g., may use simple optical character recognition (OCR) to identify, understand, and extract data from text in scanned forms). The answers to the questions in the paper form may be extracted by this service or tool of the electrophysiology database and customized TMS treatment system 200. This information may be used as context for the EEG data that may be captured and recorded for each patient.

The electrophysiology database and customized TMS treatment system 200 may provide intelligence testing as well (may also be called neuropsychiatric battery testing). This intelligence testing may be described as a brain check, which can be used, on tablets (e.g., five-minute psychiatric battery test). The electrophysiology database and customized TMS treatment system 200 may use this brain check or brain test providing additional information and coloring to the EEG data. These tests may include clinical scales, symptom profiles, and/or chief complaints for patients that may also be received by the electrophysiology database and customized TMS treatment system 200. The electrophysiology database and customized TMS treatment system 200 may use this information with the EEG data for providing diagnoses, brain health indications, and treatments for patients.

The electrophysiology database and customized TMS treatment system 200 may use clinically generated data (e.g., including labels) with EEG data for providing diagnoses, brain health indications, and treatments for patients. The clinically generated data may be received by the electrophysiology database and customized TMS treatment system 200 from a chief complaint for each patient. For example, where a patient may be diagnosed as ASD, the patient's data may put them into a diagnostic bin or folder and then the electrophysiology database and customized TMS treatment system 200 may provide a software layer or component that may request information and/or data from the user (e.g., from clinician user or patient user). In some examples, the requested information may be generally the same for each patient or may be specific (and may vary) based each patient's brain condition. The requested information may include clinical scales. In some examples, the requested information may be for the clinical scales to be inputted as individual questions or components of the clinical scale. The electrophysiology database and customized TMS treatment system 200 may provide this tool to clinics as a data dashboard so that clinician users may view status of patients (e.g., PTSD score for a patient decreases) and a place for users to input data that may be collected (e.g., clinical scales).

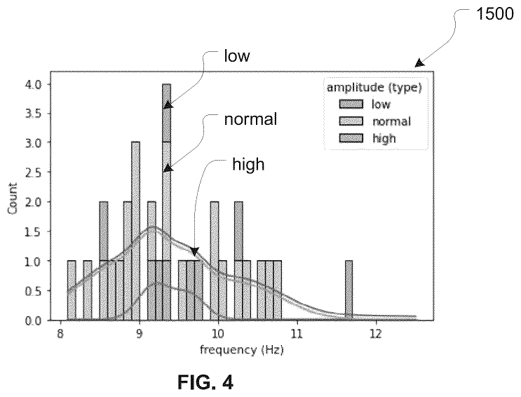

Referring now to further example implementations, FIG. 4 shows an example burst histogram 1500 for a sample EEG that may be used for determining a diagnosis of patient based on bursting characteristics. This histogram may be included in reporting (e.g., using the reporting system 206) such as an example reporting screenshot within the electrophysiology database and customized TMS treatment system 200.

Electrophysiology Database and Customized TMS Treatment System—Artificial Intelligence

The electrophysiology database and customized TMS treatment system 200 may include an electrophysiology database system having electroencephalograms (EEGs) from pre and post treatments for providing feedback in terms of diagnosis and treatment of patients such that the electrophysiology database system may use artificial intelligence (e.g., using the artificial intelligence system 224) for determining diagnosis and treatment. In some examples, the artificial intelligence (e.g., artificial intelligence system 224) may be used for determining a diagnosis for a patient. The artificial intelligence (e.g., artificial intelligence system 224) may also be used for determining and recommending the treatment for the patient. The electrophysiology database and customized TMS treatment system 200 may use the artificial intelligence system 224 for providing various applications based on machine learning. The artificial intelligence system 224 may provide neuro modulation protocols (e.g., how a patient may be treated). One modulation protocol may be placing a new patient coming in via a brain score (or where the patient may fall against other or similar clinical reports) and may relate to patients that may be coming in for performance issues. These patients may include patients that may be suffering from anxiety but they may not tell anyone, patients that may not be well focused, professional athlete patients that may be at the top but may find that they have to push a little harder than they used to, and the like. The electrophysiology database and customized TMS treatment system 200 may use the artificial intelligence system 224 to get a relative sense of where the patient may fit (as compared to other similar patients in terms of diagnosis and biographical information such as age, sex, and ethnicity) versus where the artificial intelligence system 224 may project each patient to be which may be another bucket or folder in the electrophysiology database and customized TMS treatment system 200. In some examples, the artificial intelligence system 224 may monitor metric response over time and what may be expected to occur as a response from a reporting perspective to clinicians who may be using the artificial intelligence technology. The artificial intelligence system 224 of the electrophysiology database and customized TMS treatment system 200 may be used to understand most of electrophysiologically related data and information as far as what may be important.

The electrophysiology database system of the electrophysiology database and customized TMS treatment system 200 may use artificial intelligence (e.g., using the artificial intelligence system 224) with algorithms for determining diagnosis and treatment. The electrophysiology database system of the electrophysiology database and customized TMS treatment system 200 (e.g., using the artificial intelligence system 224) may configure data from the EEGs to be used by machine learning algorithms of the artificial intelligence system 224 to determine diagnosis and treatment. The electrophysiology database system of the electrophysiology database and customized TMS treatment system 200 (e.g., using the artificial intelligence system 224) may configure data from the EEGs to be used by the artificial intelligence system 224 to analyze the EEGs with frequency-based algorithms (e.g., using machine learning) as described in the disclosure. The electrophysiology database system of the electrophysiology database and customized TMS treatment system 200 may also be configured to monitor and analyze data with an array of types of measurements, including but not limited to connectivity-type measures of coherence, clustering, mutual information, and transfer entropy for determining diagnosis and treatment. The monitoring and analysis of these other measurement types may also utilize the artificial intelligence system 224 (e.g., use machine-learning capabilities).

Electrophysiology Database and Customized TMS Treatment System—Analysis

The electrophysiology database and customized TMS treatment system 200 may include the electrophysiology analysis system 204 for providing various analytics. The electrophysiology database and customized TMS treatment system 200 may include EEG data such that most (if not every single one) of the EEG data may have over fifteen thousand data points which may be used with relatively sophisticated discriminant analysis. For example, a variety of analyses may be applied and may be utilized in terms of discriminant analysis. One second may be two hundred data points but even those two hundred data points may provide a new relation between different data points. Thus, one second of EEG may be more than two hundred data points for various measurements and when extended to ten minutes, the data points may increase to over one hundred thousand data points (e.g., about one hundred and twenty thousand data points).

The electrophysiology database and customized TMS treatment system 200 may include an electrophysiology database system having electroencephalograms (EEGs) from pre and post treatments for providing feedback in terms of diagnosis and treatment of patients and may include the electrophysiology analysis system 204. The electrophysiology database system may apply big data analytics and/or aggregated data analytics on the EEGs (e.g., using the electrophysiology analysis system 204) for determining the diagnosis and the treatment of each patient (e.g., providing electrophysiology big data analytics). The electrophysiology database and customized TMS treatment system 200 (e.g., using the electrophysiology analysis system 204) may provide a set of aggregated data analytics on the EEG data from a set of multiple patients that may be used as an input for at least one of determining a diagnosis and recommending a treatment for the patient.

Electrophysiology Database and Customized TMS Treatment System—Diagnosis and Treatment System

The diagnosis and treatment system 210 may provide diagnoses and specific treatments for mental disorders based on EEG recordings (e.g., EEG data). For example, as described in the disclosure, the diagnosis and treatment system 210 may use bursting for determining diagnoses and treatment. Once the number of bursts may be identified, there may be a frequency range of interest (e.g., alpha). Then, the diagnosis and treatment system 210 may identify the object itself for identifying frequency of bursts in its range. There may be a patient who may have a relatively large number of about 8.5 Hz bursts in a frontal region and a smaller number of about 9.5 Hz bursts in the back of the patient's brain. There may be a ratio by which about 9.5 Hz may be relatively small which may be used for determining treatment frequency. This may be different from using a Fourier transform which may look at the frequency content and note that there may be a cluster of activity that may be less specific (e.g., cluster between about 8.2 and about 8.7 Hz and there may be a cluster posterior between about 9.2 and about 9.7 Hz) such that a peak average may be about 8.3 or about 9.5 Hz which may be used as part of this decision matrix. In some examples, refining frequency elements, refining specificity, and further insights may be also be accomplished. The diagnosis and treatment system 210 may be refining from that perspective which may be one of the newer statistical approaches that may be used for identification and the diagnosis and treatment system 210 may be used to build a decision matrix based on information and data that may be received by the diagnosis and treatment system 210. For example, when referring to ASD cases, when the diagnosis and treatment system 210 may have the input of data, there may be more confidence with respect to the output of treatment. In some examples, when there are disruptions discovered in the EEG data, the diagnosis and treatment system 210 may select treatment of two places in the brain and not only one. This added experience and understanding may impact an ultimate recommendation for treatment by the diagnosis and treatment system 210 that may be transferred to the portable NEST system 100 (e.g., at the clinic) that may be executed automatically by the portable NEST system 100. This two-place treatment approach may be different from more typical treatments such as where the patient may generate health activity in their frontal lobe, the diagnosis and treatment system 210 may respond that it may be more important for the diagnosis and treatment system 210 to treat posterior regions of the brain (e.g., perieto-occipital region, parietal lobe region, and/or occipital lobe region) instead of treating frontal regions of the brain (e.g., frontal lobe region). The diagnosis and treatment system 210 may include a related matrix for the diagnosis and treatment system 210 to use. The diagnosis and treatment system 210 may also use the continuous wavelet transform application in order to identify apparent activities. Fourier transform analysis over the bursting may also be calculated.

The electrophysiology database and customized TMS treatment system 200 may include the data store (e.g., the patient recorded EEGs data store 208) or the database that may include about thirty thousand EEGs or more (e.g., having EEG characteristics that may be analyzed for a number of mental disorders). The electrophysiology database and customized TMS treatment system 200 may include a proper software environment or ecosystem. In some examples, the data store or database of EEG data may be an SQL database. A proper environment or proper place as software may be getting the EEG data into a place where the EEG may be easily queryable and may be explorable. This may utilize the object storage service (e.g., S3 bucket of Amazon AWS™ as described in the disclosure). Then, next steps may be determining what tools may be used in order to explore the data. These software tools may utilize Python-type software (e.g., general-purpose coding language) and Tableau-type software. The analyses may be translated from C or MATLAB over to Python such that it may be reasonable for any data scientist to start building analysis data pipelines. The electrophysiology database and customized TMS treatment system 200 may provide for the ability to build data pipelines such that when an EEG recording is received, the electrophysiology database and customized TMS treatment system 200 May artifact EEG data differently based on what may be seen for input of data. If a patient has autism as their tag versus head injury, the electrophysiology database and customized TMS treatment system 200 may make different decisions in terms of how the data may be handled because the electrophysiology database and customized TMS treatment system 200 may reliably expect autism cases to be less cooperative and may shake their head quite a bit during the EEG recording, causing more non-EEG noise, versus an adult patient without autism. Therefore, decisions about how the data may be processed or how sensitive the analysis of data may be to non-EEG related data may be modified. For example, seizure activity in an EEG may look similar to when a person moves their eyes left and right. Beta activity may look similar to when the patient may clench their jaw. If the patient is known coming to the clinic has a diagnosis, then the portable NEST system 100 and the electrophysiology database and customized TMS treatment system 200 may be adjusted for sensitivities to the analysis ahead of time toward building the data pipelines for different purposes. For treatment prediction, treatment frequency and location, there may be a pipeline versus data analysis pipeline against everything else. Each of these pipelines may bring with them different sensitivities. These pipelines may be built on a backbone of Python code or other suitable coding languages and systems.

Electrophysiology Database and Customized TMS Treatment System—Specific Indications and Conditions

The electrophysiology database and customized TMS treatment system 200 may use the diagnosis and treatment system 210 to provide customized TMS treatment for specific indications and conditions. The diagnosis and treatment system 210 may provide the customized TMS treatment that may include TMS based on at least one of a specific indication and a condition that may relate to a mental disorder or a condition for a patient. The provided TMS may be brain stimulation based on a level of the mental disorder or the condition for the patient. The mental disorder or the condition may be PTSD such that the diagnosis and treatment system 210 (e.g., as part of the platform of the electrophysiology database and customized TMS treatment system 200) may allow for treatment of PTSD through specific brain stimulation. The mental disorder or the condition may be a mood disorder (e.g., depression disorder), an anxiety disorder, an autism spectrum disorder (ASD), Alzheimer's, and a traumatic brain injury. For these other examples, the diagnosis and treatment system 210 (e.g., as part of the platform of the electrophysiology database and customized TMS treatment system 200) may allow for treatment of mood disorders (e.g., depression), generalized anxiety disorder, autism spectrum disorder (ASD), Alzheimer's, and traumatic brain injury through specific brain stimulations.

The diagnosis and treatment system 210 may provide the customized TMS treatment that may include TMS based on at least one of a specific indication and a condition. The customized TMS treatment may use EEGs from pre and post treatments for providing feedback in terms of diagnosis and treatment of patients. The customized TMS treatment may target the provided TMS specific to disorders and based on EEG characteristics identified in the EEGs from pre and post treatments.

Electrophysiology Database and Customized TMS Treatment System—Analyzing EEGs with Frequency-Based Algorithms

The electrophysiology analysis system 204 of the electrophysiology database and customized TMS treatment system 200 may provide for analyzing of EEG data with frequency-based algorithms. The electrophysiology analysis system 204 may provide for discovery of frequency disparities (e.g., using the artificial intelligence system 224) that may often correlate with symptoms related to cognitive challenges (e.g., PTSD and autism). This analysis may enable both automated and human driven neurological treatment planning. The EEG and TMS process 400 may include analysis/discovery of cognitive state to treatment. For example, with an autistic patient, the EEG data may be expected to have a high density of a type of activity in the posterior of the patient's brain that may not be seen in the front. This may be a relatively reliable pattern. A patient with a head injury may be expected to have a relatively high amount of slow activity in the front compared to people who may not have head injuries. The electrophysiology database and customized TMS treatment system 200 may be built as to include a database of individual EEGs with labels that may indicate that new patients having EEGs with a relatively high amount of frontal slowing needs may relate to a normal amount of frontal slowing with a person having a concussion. This observation and/or identification may indicate the treatment that may be needed. Diagnosis may be provided based on a significant amount of data that may have statistical p-values for confidence that may allow for EEG data to be placed in a head injury folder or bucket where EEG data may have patterns associated with patients having head injuries as described in the disclosure. As the p-values improve, then it may be possible to obtain FDA approval on diagnoses. At the very least, the electrophysiology database and customized TMS treatment system 200 may be set up such that there may be these parameters and these frequency boundaries where if a peak value alpha frequency may be under about eight Hertz. Where the patient may be older, there may be some dementia that may worth observing related to Alzheimer's diseases. In this example, the electrophysiology database and customized TMS treatment system 200 may use EEG data and associated patterns of EEG data for diagnosing Alzheimer's, dementia, and/or Parkinson's disease. As the data store and/or database of the electrophysiology database and customized TMS treatment system 200 may improve in terms of quantity and quality of EEG data, there may be increases in statistical confidences such that each patient may fit into a cluster. If you combine the associated EEG data with a few clinical symptoms, the diagnosis itself may be supportive. The electrophysiology database and customized TMS treatment system 200 may use the addition of other data (e.g., clinical data) in combination to help provide context for the EEG data in making diagnosis determinations.

There may be algorithms used with historical data as part of this analysis. The electrophysiology analysis system 204 may use mathematical algorithms for identifying pertinent features. The electrophysiology analysis system 204 may involve building more with a first measurement that may be an additional measurement, which may be input variables. The electrophysiology analysis system 204 may involve weighting as part of the algorithms. Given input measurements (e.g., age of the patient), if there are measurements at about 8 Hz and another measurement at about 10 Hz, then enough information may be available to define and determine a state of the patient. For example, a decision matrix may be used with clinicians such that reliable algorithms that may be in existence may be captured in a programmatic sense. These algorithms may be based on correlations between components of EEG data that may be associated with a mental disorder (e.g., autism in children). When there is, an observation of a component that may be well correlated with what may be seen in a traumatic brain injury (TBI); the electrophysiology analysis system 204 may capture this algorithm because the input and output may be used for clustering. The electrophysiology analysis system 204 may use an algorithm that may include weighting variables by location that may output a number value for treatment. These weights may be adjusted for intermediate steps to make sure outputted number values may be reliable.

The electrophysiology analysis system 204 may use technology for analyzing a cognitive state (e.g., use the artificial intelligence system 224). The electrophysiology analysis system 204 may analyze EEG data and may derive deep insight into each client's brain health such that the analysis may combine expertly honed algorithms with historical data (e.g., years of data) to create an actionable cognitive report (e.g., using the reporting system 206). For example, identification of principal EEG spectral components and identification of activities, by region, may form the baseline EEG profile that may inform therapy approach and expected responses. With this analysis, frequency disparities may be discovered by the electrophysiology analysis system 204 that may often correlate with symptoms related to cognitive challenges (e.g., PTSD and Autism). The electrophysiology analysis system 204 (e.g., using the artificial intelligence system 224) to understand these disparities may help patients and clinicians in determining the best strategy in terms of current and future treatment.

The expertly honed algorithms may be based on a variety of factors and variables. For example, included in the algorithms may be rolling windows that may adaptively scale to capture and classify specific wavelets versus background noise, with a possibility for integration of wavelet transforms during preprocessing for further optimization. At this stage in the process, wavelet validity may be tested with nearest-neighbor coherence and transfer entropy measures counted above preset thresholds. Relative EEG power distribution may be determined from identified wavelet ‘objects’ distribution and identification of principal EEG components that may contribute to the generation of the EEG profile, or ‘type’, which may be connected to sensitivity or resistance to therapeutic response, thus triggering adjustments to therapy. Some EEG types may include low voltage cases requiring multiple locations of stimulation, or focal slowing due to stroke that may require local and lateralized stimulation based on identification of functional deficit foci, or identification of EEGs via burst analysis where bursts may remain scattered after the first few EEGs that may require additional treatment locations. Identification of multiple alpha cortical generators may be more resilient to therapy and may require posterior stimulation over anterior stimulation.

As it pertains to cognitive reports, specificity may be brought through the identification of patient ‘brain type’ via specific predetermined characteristics. In addition, machine-learning techniques may be applied such as k-means clustering and principal component analysis as well as definition by a specific variable with predefined measurement cutoffs. For example, an anterior-posterior alpha band amplitude ratio may be combined with a regional frontal coherence threshold for identification of patients with frontal alpha generation. Given the identification of EEG subtype, patients may be compared to an average representative of similar brain types, as collected by the EEG system (e.g., as part of the EEG and TMS process 400). This relative comparison may provide a relatively more accurate measure of individual ‘cognitive network’ health versus a comparison of the patient versus an average from all EEG profiles.

Additional insight into the network health and subsequent cognitive state may be through an application of a relative brain synchrony index (RBSI) where an ideal frequency of function may be identified. The identified ideal frequency of function may differ from peak alpha frequency and a comparison between current peak alpha frequencies versus ideal frequency may help gauge an overall reduction in function versus ideal. Therefore, cognitive capacity versus individual ideal in addition to capacity for response to treatment and possibly overall duration of treatment may be determined. Through utilization of adaptive peak identification algorithms, a patient may have a dominant EEG alpha at about 8.7 Hz, with a reduced but present alpha at about 9.8 Hz, and at about 10.6 Hz. This analysis may indicate a conclusion that the patient may be about two degrees from ideal peak alpha frequency and therefore working at relatively reduced capacity and prime for response to neuromodulation. A patient may have a dominant spectral frequency of about 7.5 Hz frontally, and a posterior dominant alpha rhythm at about 8.5 Hz, with evidence of rhythmic alpha bursting at about 9.3 Hz and about 10.2 Hz. This patient may be considered more severely impacted clinically due to the reduction of spectral activity below the cutoff for alpha activity (e.g., about 8.0 Hz cutoff) and due to a separation from what may be ideal peak frequency of about four or more rhythmic EEG steps.

The EEG and TMS process 400 may utilize other types of algorithms. The EEG and TMS process 400 may utilize Hilbert transforms, hamming windows, and/or different variations on mother wavelets (e.g., Daubechies (db4), Morlet, etc.). In some examples, progressively narrowing band pass filters may be used around detected peak frequencies. For example, if a peak frequency of about 11 Hz is noted, there may be multiple ordered filters, such that a first order may be from zero (0) to twice a detected frequency, the second order may be a set percentage from zero (0) and the detected frequency and correspondingly reflected on the faster range, etc. These progressive filters may indicate cortical organization by their ratios, or relative network health by their interrelations.

In terms of data depth, frequency may be particularly focused on for analysis. The electrophysiology analysis system 204 may analyze for following informative data: relative burstiness and amplitude of a burst, how many wavelets may be in a burst, how each burst may move through space, etc. This informative data may be additional input measurements used by the electrophysiology database and customized TMS treatment system 200 (e.g., for analysis by the electrophysiology analysis system 204 and for diagnosis and treatment by the diagnosis and treatment system 210).

When different regions of the brain may share a same wave pattern, the brain may be more efficient. When regions are not as synchronized, cognitive symptoms may be observed. For example, when a client has depression, their alpha and theta wave patterns may typically differ across left and right frontal hemispheres. The electrophysiology database and customized TMS treatment system 200 may provide for collection of historical data that may allow for correlation of unique patterns to symptoms and mental conditions.

Electrophysiology Database and Customized TMS Treatment System—Diagnosing and Targeting TMS Treatments

The diagnosis and treatment system 210 may provide diagnosing and targeting TMS treatments that may be specific to disorders and may be based on EEG characteristics from the electrophysiology database and customized TMS treatment system 200. The diagnosis and treatment system 210 may provide targeted, individually designed, treatment based on specific patient frequency patterns. In examples, the diagnosis and treatment system 210 may provide targeted treatment based on patient frequency patterns and/or based on disparity approaches. The diagnosis and treatment system 210 may use this process with the decision matrixes as described in the disclosure. For example, where a patient comes in with a stroke and their EEG data may be labeled as stroke, there may be a stroke related TMS treatment that may be determined and customized for each patient. For example, assuming a stroke on the left side of the patient such that function in EEG data may be slower on the left side, the diagnosis and treatment system 210 may recommend treatment to the left side of the patient's brain (e.g., same side as the stroke side of the brain) such that the treatment may strengthen the left side function of the patient's brain and help the patient deal with disparity in terms of frequency due to a frequency disparity between left and right hemispheres. In summary, as may be contrary to industry standards, the side of the patient's brain that may be damaged tissue may be treated (e.g., as determined and recommended by the diagnosis and treatment system 210). In the diagnosis and treatment system, 210 may recommend strengthening of source generation and treatment of the same side of damaged brain tissue (e.g., stroke side of brain) as well as recommended treatment of regions downstream of stroke foci that may be impacted and slowing. The diagnosis and treatment system 210 may provide an approach that may treat the disruption because of the injury and may try to strengthen the patient's brain from a source of frequency generation. This treatment decision may be based on stroke treatment (e.g., as identified from EEG data labeling) and may be based off the EEG data captured. The diagnosis and treatment system 210 may determine treatment based on degree to which there may be a stroke influence, degree to which the patient's brain may be slowing such as with a severe stroke vs. a less severe stroke, etc. Another factor that may affect treatment may include frequency information of impacted region versus area of the brain that may not have been impacted by the stroke. This stroke example may be an example where a frequency disparity being used may be based off of an input variable of the diagnosis where the diagnosis and treatment system 210 may determine treatment of the area that may have the frequency disparity or the disruption. When it comes to frequency disparities, in some examples, there may be a frequency disparity between about 9 Hz and about 11 Hz. The density of the frequency itself of the EEG data may be another dimension used by the diagnosis and treatment system 210. In an example, there may be about ten microvolts of about nine microvolts in the back portion of the patient's brain for an 11 Hz frequency and there may be about two microvolts of about nine microvolts in the front portion of the patient's brain for the same 11 Hz frequency. The diagnosis and treatment system 210 may determine if the disparity within the frequency may be expected or if the ratio may be too low. Generally, the diagnosis and treatment system 210 may determine whether the ratio may be too low (e.g., about nine microvolts in a back area of the brain and about four, four and a half, five microvolts in the front of the brain). In some examples, there may be a disparity for that frequency between regions such that diagnosis and treatment system 210 may recommend proceeding with treating the region that may be responsible for generating the frequency where this frequency may be on the left hemisphere or right hemisphere (e.g., a location may be at a specific quadrant of the right or left hemisphere). In addition, the diagnosis and treatment system 210 may monitor and/or look for the region where there may be the largest deviation in that frequency by density globally (which may tend to be a frontal lobe). In some examples, the diagnosis and treatment system 210 may determine and recommend treatment of multiple locations that may be specific to where the largest deviations may be occurring.

Where there may be a disparity on the front and the back of the patient's brain, the diagnosis and treatment system 210 may recommend application of neuromodulation towards the front, which may affect and improve EEG signals in the back of the patient's brain. For example, if the diagnosis and treatment system 210 may apply a treatment more towards the front, there may be an impact and improvement on the EEG data recorded and the disparities in the back of the brain, and vice versa. The diagnosis and treatment system 210 may recommend treatment on the front of the brain to impact/improve disparities in back of the brain, and vice versa. In some examples, the EEG and TMS process 400 may provide treatment to regions responsible for the brain weaknesses in frequency. The EEG and TMS process 400 may include applications of TMS to the front of the brain such a change may occur towards back of the brain before there may be any changes to the front of the brain. In general, depending on what the patient may be coming in with, the diagnosis and treatment system 210 may make additional determinations and measurements as compared to treatments that may have worked well or not as present in the database, and as compared to patients who may have come in with the same report but not necessarily the same degree of difference.

Usually the rhythm that may be treated may be in the back of the brain (on either the left side or the right side) and this treatment may ring through the patient's brain. There may be a general industry standard to find a brain structure (e.g., all dorsolateral prefrontal cortex (DLPFC or DL-PFC)) that may have a relation with the patient's midbrain and then may try to stimulate or lower the behavior of that brain structure without any frequency related content that may be related to EEG data. The electrophysiology database and customized TMS treatment system 200 (as part of the EEG and TMS process 400) may try to restore the health of a posterior to anterior network. This may involve applying treatment to the region that may have the least amount of activity. Treatment may be provided locally to the region that may be stimulated to provide ringing.

The electrophysiology database and customized TMS treatment system 200 may provide an optimized approach that may be most comfortable. These treatments may be more sensitive to children that may treat their cortical function. The electrophysiology database and customized TMS treatment system 200 may have a different decision, e.g., by age or by diagnosis. In some examples, if a patient has a stroke, the electrophysiology database and customized TMS treatment system 200 may provide a different decision in terms of the locations that may be treated.

The electrophysiology database and customized TMS treatment system 200 (specifically the diagnosis and treatment system 210) may provide for targeted treatment based on unique transcranial-cranial frequency patterns of each patient. The diagnosis and treatment system 210 may suggest stimulation of neurons that may be targeted regions of brain training neurons to fire more synchronously throughout the brain (e.g., as executed portable NEST system 100). If disparities may be identified by the diagnosis and treatment system 210, the diagnosis and treatment system 210 may develop a targeted treatment (e.g., using MyWaveTMS) that may be tailored to a patient's unique frequency pattern that may use TMS. As described in the disclosure, the TMS may be a non-invasive treatment that may improve synchrony of brain waves. TMS may use an electromagnetic coil that may stimulate neurons in targeted regions of the brain, which may gradually train neurons to fire more synchronously throughout the brain. In some examples, specific mental disorders may require targeted treatments constructed based on EEG data such that styles of research may be applied for any kind of mental disorder when there may be enough or sufficient data.

The electrophysiology database and customized TMS treatment system 200 (specifically the diagnosis and treatment system 210) may be used to diagnose mental disorders. In some examples, this diagnosis may be based on an average of patterns or identified common disruptions noted in other patients with the same particular mental disorder. There may be expected patterns or disruptions of function for patients that may have PTSD. The diagnosis and treatment system 210 may provide training for clinicians to understand a degree to which the clinicians may have to improve the patient's EEG and how far off the patient's EEG may be with respect to what may be considered normal EEG data for similar patients. With the input of the Fourier transform and the density (or the amount of these different frequencies of activity), such that the diagnosis and treatment system 210 may have a relatively confident understanding of the patient for when new EEG data may be received on the patient or similar diagnosis may be made for another patient having similar EEG data.

The diagnosis and treatment system 210 may use EEG data from databases as support tools for treating mental disorders with TMS. For example, brain monitoring may be combined with automatic analysis of EEGs that may provide a clinical decision support tool that may reduce time to diagnosis and assist clinicians in real-time monitoring applications (e.g., neurological intensive care units). In some examples, clinicians may have indicated that a sensitivity of about 95% with specificity below about 5% may be the minimum requirement for clinical acceptance.

The diagnosis and treatment system 210 may provide a systematic approach to targeted treatment which may be slightly adapted for each mental disorder. For example, one trait may be that children may not have dominant rhythms generally as fast (e.g., the intrinsic frequency of an EEG band may have a lower value) as adults. For patients with dementia and Alzheimer's, there may be cortical tissue loss and these patients may not be as fast as if they had the cortical tissue intact. In another example with Autism, the diagnosis and treatment system 210 may direct an application of stimulation to multiple regions immediately. However, in other examples with PTSD, the diagnosis and treatment system 210 may wait two to four weeks to account for therapy response sensitivity. With bipolar disorder, the diagnosis and treatment system, 210 may recommend the power of the stimulation not be as high (e.g., about 60% of motor threshold in terms of the amplitude) because the simulation may impart with it dopaminergic increases and patients with bipolar disorder may be particularly sensitive to the dopaminergic. The diagnosis and treatment system 210 may adjust treatment (e.g., limit power of treatment in some circumstances and/or adjust other treatment parameters) depending on mental disorder being treated as well as age, sex, ethnicity, etc. of the patient.

The electrophysiology database and customized TMS treatment system 200 may provide a software design that may have various fields. Algorithmically, the software may populate these fields. For example, one field may be a motor threshold field; another field may be the recommended percent of treatment output intensity field, etc. In some examples, fields may be related to mental disorders and/or conditions that may affect treatments.

Electrophysiology Database and Customized TMS Treatment System—Predictive Analytics

The electrophysiology database and customized TMS treatment system 200 may include the predictive analytics system 222 that may be used to identify people before they get ill and may provide treatment and/or care recommendations based on the electrophysiology database and customized TMS treatment system 200 (e.g., specifically patient recorded EEGs data store 208). In some examples, insights may be obtained from multiple patients for predicting what may be triggering mental disorder (e.g., not enough sunlight for depression). In some examples, EEG frequency content, distributions, and different bands for patients who may be healthy and may have no disruptions may be obtained such that there may be different normal resting EEG patterns and densities (e.g., as relating to different brain types). In some examples, when EEGs may be measured for patients with their eyes closed, the EEG recorded data may have an excess of beta activity or working brain function whereas most patients may have lower density beta EEG data during eyes closed. The electrophysiology database and customized TMS treatment system 200 may identify this beta activity and indicate possibility for anxiety disorders. By measuring this relevant data, the diagnosis and treatment system 210 may identify patients as having anxiety (e.g., patient may be predisposed to anxiety). With regard to some of the EEG patterns observed, the diagnosis and treatment system 210 may look to increases in low frequency EEG activity during eyes-closed resting states and compare this data versus what may be expected for the same condition and similar populations. The electrophysiology database and customized TMS treatment system 200 may identify a density of slow-wave activity as abnormally high and may indicate a possibility for cognitive slowing or attention interference that may be corrected. The RBSI may be applied to indicate when dominant spectral rhythms globally may be slowing as compared to what may be considered ideal for that patient, and may indicate that cognition is declining and treatment may be warranted. There may be an ideal EEG type of template that may be used as a reference for determining treatment (e.g., by the diagnosis and treatment system 210). In some examples, metrics or patterns may imply positive or negative.

In general, different brain types may have pros and cons (e.g., strengths and weaknesses) which may be due to resting rhythms contributing to either more or less sympathetically activated neuronal systems (e.g., parasympathetically). Some brain types may be better at focusing in on single details than others. There may be personality and performance characteristics associated with different brain types. In terms of performing treatment, the diagnosis and treatment system 210 may determine and suggest treatments that may try to lower theta power, try to change beta power, try to adjust other parameters, etc. These parameter improvements may relate to achieving idyllic EEG data. This may be where clustering may be used by the diagnosis and treatment system 210 (e.g., also using the electrophysiology analysis system 204). For example, if the diagnosis and treatment system 210 identifies a patient's brain type, the diagnosis and treatment system 210 may then compare the patient's brain to other patients with the same brain type (e.g., using the electrophysiology analysis system 204) and the diagnosis and treatment system 210 may determine to what degree there may be an addition of activities as disruptions. Then, the diagnosis and treatment system 210 may indicate that there may be a need to address the activities to bring brain status to a relative point that may be ideal for the patient. From a predictive analytics perspective, the predictive analytics system 222 may expect specific brain types to have predispositions towards different mental disorders or to be more sympathetically or parasympathetically activated such that the diagnosis and treatment system 210 (e.g., using the predictive analytics system 222) may provide feedback and then the additional perspective of frequency may be added (e.g., comparing slow frequency to where the frequency ought to be, as an ideal from which departure may signal a warning flag).

The electrophysiology database and customized TMS treatment system 200 may determine from EEG data that some people may have a relatively higher rate of depression given that these people may not have enough sunlight (e.g., using big data analytics and/or aggregated data analytics with predictive analytics). Thus, the electrophysiology database and customized TMS treatment system 200 may want to provide an outreach to promote certain types of wellness behaviors. Further, the predictive analytics system 222 of the electrophysiology database and customized TMS treatment system 200 for predicting what may be triggering a mental disorder (e.g., not enough sunlight for depression). The predictive analytics system 222 may consider outside external factors for determining what may be triggering specific mental disorders in addition to the mental disorders themselves. The predictive analytics system 222 may monitor at how data may be handled such that the more information available may provide context to the data, which may be used to make determinations from the data. Information related to context may include disrupted sleep, limited sunlight, history of head injury, opiate use or drug use of any kind, a stroke, etc. This may provide context on why different factors in the EEG data may not normally be there and why other external factors that may relate to a clinical biography of the patients may be affecting EEG data results. In general, there may be brain functions that may be disruptions from ideal function and there may be ideal function. Having an extended period without appropriate morning sunlight and disrupted sleep may reinforce that disruption. In some examples, too much of that disruption may push patients towards what may be considered and classified as a mental disorder or a disruption in the normal brain function to the extent where it may cross some clinical boundary. The electrophysiology database and customized TMS treatment system 200 may have an algorithm that may take as input, for example, living in places such as Seattle or Norway may have limited sunlight for half the year, or a veteran or an active duty person may suffer from shell shock and other forms of post-traumatic stress disorders. This type of information may be provided to trained clinicians, which may be provided as input when interpreting the EEG data. The electrophysiology database and customized TMS treatment system 200 as a platform that may have these types of data fields as input against which these types of analysis may be conducted. The electrophysiology database and customized TMS treatment system 200 may obtain contextual information associated with the EEG data useful for analysis and making decisions by various systems and components (e.g., used by the electrophysiology analysis system 204, the reporting system 206, the diagnosis and treatment system 210, the treatment modelling system 220, the predictive analytics system 222, and the artificial intelligence system 224).

The electrophysiology database and customized TMS treatment system 200 may allow for inputting from a data stream view (e.g., data that may be coming from clinician meetings may be inputted to the system 200). There may be a patient registration. If the patient shows up at any clinic, the patient may be registered and there may be a chief complaint field that may be filled out and submitted to the electrophysiology database and customized TMS treatment system 200 for the patient. The chief complaint field may include information such as the patient may have autism, the patient may have a history of head injuries, or the patient may have anxiety. The chief complaint may also include date of birth, gender, and other biographical information, which may be received from the patient information field. The clinical data that the clinician supplies to the electrophysiology database and customized TMS treatment system 200 may include clinical data that may be gathered. The electrophysiology database and customized TMS treatment system 200 may request for the data after the clinician meeting (e.g., when reviews may be completed). The electrophysiology database and customized TMS treatment system 200 may be built as a software infrastructure that may automatically capture as a front end as a service for clinicians. For example, use of the electrophysiology database and customized TMS treatment system 200 such that patients may be able to have clinical scales easily accessible which may provide additional insight on the EEG data. This associated information may be tracked and tagged with EEG data appropriately.

The electrophysiology database and customized TMS treatment system 200 (specifically the predictive analytics system 222) may utilize prodromal or predictive medicine where populations may identify in a preventative manner based on EEG data before they develop (e.g., using big data analytics and/or aggregated data analytics with predictive analytics). The predictive analytics system 222 may identify and outreach to higher health risk populations based on EEG data in a database and may encourage the patients to seek medical care depending on studies (e.g., there may be a higher rate of cervical and dermatologic cancers in one region over other regions in the same country that may also be associated with higher rates of depression). The predictive analytics system 222 may identify and outreach to populations in a preventative manner before the patients may develop cancer or heart disease based on EEG data in the electrophysiology databases (e.g., of the electrophysiology database and customized TMS treatment system 200). The predictive analytics system 222 may use data and information (e.g., including contextual information) in the electrophysiology database and customized TMS treatment system 200 to identify patients before they develop, for example, dementia or Alzheimer's disease. In addition, the predictive analytics system 222 may use EEG data (e.g., electrophysiology databases of the electrophysiology database and customized TMS treatment system 200) to identify patients who may have a certain type of fragility (e.g., more prone to PTSD, which may include applications for military, police officers, firefighters, etc.). The predictive analytics system 222 may identify populations and/or patient groups based on EEG data and other information in the electrophysiology database and customized TMS treatment system 200 for providing insights and/or predictions for these identified populations and/or patient groups.

There may be disruptions that may normally develop and may increase. These disruptions may be related and/or associated with brain types as described in the disclosure. There may be different bands of brain activity such as from slow to fast, delta theta on the slow side of the brain, alpha beta and some gamma on the faster side of the brain, etc. In some examples, patients may have more beta activity than other patients. Brains may have high amounts of beta activity. It may be normal to have some beta activity but there may be heterogeneity in the population such that some patients may be predisposed towards having more beta activity. There may be more alpha or theta or different densities in these relationships. During resting state, where some patients may be observed to have more elevated beta activity compared to expected norms, these patients may have a predisposition towards anxiety, feeling restless, or being more sympathetically activated, which may push patients towards an anxiety class of disorders. The electrophysiology database and customized TMS treatment system 200 may provide a prediction that assess how well this anxiety may be kept in check. For example, whether kept in check by the density of different brain rhythms, which may be in the alpha, range (brain clock rhythm) or may be kept in check when it comes to lifestyle habits. The electrophysiology database and customized TMS treatment system 200 may provide a brain score or a brain metric that may score how much beta or restlessness patients may have which may be used against a daily log to determine if the patient may be at risk. The patient may be more at risk for anxiety disorders or PTSD than patients who may not have high amounts of beta activity. This type of correlation may relate to any of the anxiety classes.

There may be patients that may have elevated alpha rhythms when eyes closed (e.g., higher density and in different regions than where it may be expected). Some patients may generate these alpha rhythms frontally whereas a majority of patients may generate these rhythms posteriorly. In some examples, the electrophysiology database and customized TMS treatment system 200 may notice from notes or other information that patients may have too many parasympathetic behaviors/responses (e.g., “rest and digest”), too little sympathetic behaviors/responses (e.g., “fight or flight”), and relatively low activated brains may be diagnosed as having depression or low energy/mood. From this information and/or data, the electrophysiology database and customized TMS treatment system 200 may cluster to provide an indication as to the patient being predisposed.

The electrophysiology database and customized TMS treatment system 200 may monitor and provide analysis for relative brain synchrony (RBS) size (e.g., this may be a shortened version of the relative brain synchrony index (RBSI)) where the electrophysiology database and customized TMS treatment system 200 may refer to frequency or speed of the brain clock. The frequency or speed of brain clock may be referred to with respect to therapy protocols. The electrophysiology database and customized TMS treatment system 200 may choose a frequency or may calculate the frequency. This frequency may be typically in the alpha range. The frequency may not be in the alpha range because of the brain clock activity. As patients age and grow, the patient's may speed up to a certain frequency and may slow down normally with time. People's frequency or speed may typically slow down eventually.

The electrophysiology database and customized TMS treatment system 200 may check a degree to which a patient may be slowing down relative to ideal for the patient. With an ideal frequency, the electrophysiology database and customized TMS treatment system 200 may calculate a patient is zero point. Interesting insights may be discovered such as the patient may tend to slow down in about 0.8 to about 1.2 Hz jumps that may seem to correspond with heart rate frequency. If a patient may be at about 11.3 Hz, the electrophysiology database and customized TMS treatment system 200 may observe activity at about 10.2 Hz and about 9.1 Hz, for example. If a patient may be at about 9.6 Hz, the electrophysiology database and customized TMS treatment system 200 may observe frequency over 10.5 Hz. This may correspond to notes such as if the patient may be at about 11.3 Hz, the patient may be at zero point. In addition, the electrophysiology database and customized TMS treatment system 200 may determine 11.3 Hz as a posterior rhythm that may occur about twenty percent of the time. In the same region, the electrophysiology database and customized TMS treatment system 200 may observe a posterior rhythm at about 10.1 or about 10.2 Hz that may occur about sixty percent of the time. In some examples, the electrophysiology database and customized TMS treatment system 200 may make the postulation that the patient should be up at about 11.3 Hs instead of being down at about 10.2 Hz. This may be one note or an F1 difference from ideal. The greater the differential may be, if down two notes slower or three notes slower, the more disruptive the patient may be from ideal and the more at risk the patient may be may which may lower the patient's relative brain score. When the frequency is about eight or sub-eight Hz frequencies, there may be possible memory loss or memory disruption, elevated fatigue, and other symptoms that may be seen more commonly in Alzheimer's or Parkinson's patients. If patients have a major traumatic brain injury, the patients may immediately move from either F01 down to an F4 or F5. If the patient has a stroke, it may be quite traumatic and may be relatively difficult for the patient's brain to organically move itself back up in any short or relative period of time without any intervention. The electrophysiology database and customized TMS treatment system 200 may be used to analyze alpha rhythms such that the fast Fourier transform (FFT) may look at frequency, speed, or number of oscillations per second. For younger patients, frequency may be typically faster whereas with older patients, frequency may be slower. This may relate to power increases (e.g., power may drop and may then increase again) where amplitude may decrease and may resynchronize faster. In general, patients with faster brains may have improved working memory (e.g., this may be based on lining up of alpha peak frequency versus working memory showing positive correlation). In some examples, the electrophysiology database and customized TMS treatment system 200 may determine a projected ideal speed for each patient. This ideal speed or rhythm may be generated posteriorly but may occur globally.

The electrophysiology database and customized TMS treatment system 200 may observe on the development delay side that the patient may have a developmental delay vs. autism. The electrophysiology database and customized TMS treatment system 200 may make discriminations between EEG data and clinic reviews for providing indications of diagnosis. There may be kid patients that may be listed as being diagnosed with autism but from analysis of EEG data and other information, the electrophysiology database and customized TMS treatment system 200 may determine that the kid patients may have developmental delays not autism. The characteristics on the symptom profile may be similar between autism and developmental delay (e.g., delayed language sensory processing awareness). EEG data and other tests (e.g., genetic testing) may assist the electrophysiology database and customized TMS treatment system 200 in distinguishing diagnosis between autism and developmental delay.

The electrophysiology database and customized TMS treatment system 200 may be used with a patient having a head injury (e.g., concussion). The patient may present classic concussion syndromes and the patient's EEG data may demonstrate focal slowing in the theta range both at an injury site and possibly in other sites correspondent to the injury. Neuromodulation may be recommended and applied for about two weeks, which improves the patient's brain status and score(s). In some examples, neuromodulation may be applied at about 10.3 Hz. After applying the stimulation, there may be entrainment because of stimulation and resultant of about 10.3 Hz may be observed where previous slowing occurred. When the magnetic field may be applied at a repetitive rate, the magnetic field may produce neural entrainment that induces charge locally and influences the firing rates of underlying cortical tissue and local field potential. The resultant entrainment may affect resting network function such that identified focal theta may dissipate with treatment and the entrained frequency emerges.

The electrophysiology database and customized TMS treatment system 200 may suggest a few strategies in treating (e.g., may be executed and/or implemented by the portable NEST system 100). One example may be a reinforcement of ideal function local to the site of generation. Another example may be treating function where it may be at the greatest deficit. A combination of both may be recommended and executed.

For applications involving stroke, the electrophysiology database and customized TMS treatment system 200 (and specifically the diagnosis and treatment system 210) may determine treatment for the stroke patient. For example, where the stroke patient may have an injury on their left side, the electrophysiology database and customized TMS treatment system 200 may recommend treatment on the right for the stroke. The electrophysiology database and customized TMS treatment system 200 may recommend treatment of the injured tissue (e.g., based on the identification of an injured area of the brain). The injured region may be shown to have a slowing effect especially compared to non-injured regions of the brain. The electrophysiology database and customized TMS treatment system 200 may discriminate between these regions as far as what may be injured tissue or tissue slowed down by several notes. The diagnosis and treatment system 210 may determine and suggest target treatment based on the brain's function.

The electrophysiology database and customized TMS treatment system 200 may include the electrophysiology database system that may apply big data analytics and/or aggregated data analytics on the EEGs (e.g., using the predictive analytics system 222, the electrophysiology analysis system 204, and/or the diagnosis and treatment system 210 in any combination) for determining the diagnosis and the treatment of each patient (e.g., providing electrophysiology big data analytics and/or aggregated data analytics) with respect to predictive analytics. The electrophysiology database system (e.g., using the predictive analytics system 222) may generate predictive analytics based on the big data analytics and/or aggregated data analytics that may be used to identify patients and provide appropriate treatment for the identified patients based on the EEGs (e.g., EEG data). In some examples, the electrophysiology database and customized TMS treatment system 200 may generate predictive analytics based on the aggregated data analytics (e.g., using the predictive analytics system 222) that may be used to identify patients and provide appropriate treatment for the identified patients based on the EEG data.

Electrophysiology Database and Customized TMS Treatment System—Stem Cell Treatment Recommendations

There may be a stem cell treatment process that may include applying stem cells to improve connectivity of neurons in the brain of a patient. As described in the disclosure, this stem cell treatment process may be recommended by the electrophysiology database and customized TMS treatment system 200. Use of the stem cell treatment process may be part of the EEG and TMS process 400. In further examples, the stem cell treatment process may be part of the portable NEST system 100 and/or the electrophysiology database and customized TMS treatment system 200 in any combination. The stem cell treatment process may be used by patients where connectivity may not be present or possible for existing neurons and networks. The stem cell treatment process may be used to create new neurons where there may be a lack of neurons in terms of connectivity. The stem cell treatment process may be used to enhance stem cells. The stem cell treatment process may adjust one or more parameters based on feedback of gene expression for the neurons.

The electrophysiology database and customized TMS treatment system 200 may recommend stem cell treatment. This type of treatment for the patient may be simulated (e.g., by using the treatment modelling system 220). Stem cell treatment may relate to improving connectivity with existing neurons where the connectivity may be damaged. Even with new neurons, connectivity may still need improvement. This treatment may be used to enhance stem cells.

The stem cell treatment (e.g., as a specific suggestion by the electrophysiology database and customized TMS treatment system 200) may be gene expression. In some examples, TMS may be utilized in combination with stem cells. There may be a combination effect such that stem cells may be activated locally with stimulation. Depending on the stimulation delivered to in-vitro tissue, such as delivering one set of parameters with different frequencies and timings of stimulation versus another set of parameters with the same or different frequencies and timings of stimulation in different blocks, there may be resultant differential gene expression. There may be a change depending on frequency and dose parameters behind a stimulation.

There may be other approaches with or without TMS that may result in different gene expression for each separate approach. Determining the approach to stem cell treatment may involve honing in such as through appropriate testing in a laboratory on a specific protocol that may be helpful for a specific disorder that may have a disruptive gene expression. Each treatment may be determined and may be provided specific to each treatment profile (e.g., relating to each patient and/or each disorder/condition).

The stem cell treatment may involve CRISPR gene editing (e.g., CRISPR modifications). There may be a specific treatment that may be preferred for application and the patient may not be genetically predisposed to it such that there may be an opportunity for a gene expression CRISPR combination. For example, it may be determined that specific tissues may be causing seizures because the tissues may be genetically different. EEG data may be used to focalize seizure activity and then CRISPR may be utilized to treat and attempt to repair functions of the tissues needing repair. This may be another intervention approach.

Electrophysiology Database and Customized TMS Treatment System—Other Treatment Recommendations

The electrophysiology database and customized TMS treatment system 200 may provide diagnosis and treatments (e.g., using the diagnosis and treatment system 210) that may be related to neuro-transmitter density changes. For example, if the electrophysiology database and customized TMS treatment system 200 may determine that there may be an increase in serotonergic expression, a recommended treatment may be to lower selective serotonin re-uptake inhibitors in combination with treatment as an intervention. Other treatments may include suggested medications that may be made more powerful by utilizing modulation in different ways to reinforce and change gene expression to make it such that use of medication to lower dosage to achieve the same response or results that may not be achieved at any dosage.

The electrophysiology database and customized TMS treatment system 200 may also suggest medications such as anti-depressants for use with or without the portable NEST system 100. It has been shown that anti-depressants may have one or more neurogenic properties. Neurons may be created in the brain. However, these created neurons may not be connected effectively to other neurons. One of the advantages of the described targeted TMS therapy is that it may increase neuronal connectivity. Further, the targeted TMS therapy may be combined with anti-depressants (e.g., selective serotonin reuptake inhibitors (SSRIs), serotonin and norepinephrine reuptake inhibitors (SNRIs), etc.) to provide improved results in treating patients.

Electrophysiology Database and Customized TMS Treatment System—Reporting based on EEG Data

The electrophysiology database and customized TMS treatment system 200 may generate reporting (e.g., using the reporting system 206) based on EEG data of the patient recorded EEGs data store 208. The reporting system 206 may generate reports that may reflect current brain state and general brain health. The patient may go to a clinic to have EEGs recorded for their brain. The recorded EEGs may be uploaded to servers, and the reporting system 206 may generate a report based on the uploaded EEG that may point or refer to several characteristics of the EEG itself related to the individual.

The electrophysiology database and customized TMS treatment system 200 may be used to find disruptions where there may be elevated data in any region or a slower peak alpha frequency compared to ideal. The electrophysiology database and customized TMS treatment system 200 may point out characteristics that may be expected to be normal for a patient that may color personality (e.g., elevated beta activity or frontal alpha generators or a peak frequency that may be relatively fast, existence of multiple peak alpha frequencies, reductions in frontal alpha density compared to posterior compared to normal, or low voltage EEG). The electrophysiology database and customized TMS treatment system 200 may look at ratios between alpha and beta density, or theta and alpha densities for determining predispositions towards attention deficit disorder (ADD) if there may be an elevation of the slow wave activity or hyperactivity where there may be a slight increase for activity. These types of characteristics in addition to any and every other calculation may be compared to the norm.

The electrophysiology database and customized TMS treatment system 200 may determine local brain type (e.g., using a relatively large database) such that each patient's brain may be compared to others who may also be generators or compared to others who may also have relatively high beta activity such that the patient's brain may be compared to these groups. The electrophysiology database and customized TMS treatment system 200 may be used to compare the patient's brain to a local cluster to see where the patient's brain falls and may find the patient's median average for all activities as another comparison. Clustering may be done using share nearest neighborhood, k-means, KNN, machine-learning clustering, etc. The k-means may be preferred for clustering.

The electrophysiology database and customized TMS treatment system 200 may provide outlier management (e.g., identification of outliers). The electrophysiology database and customized TMS treatment system 200 may look at an artifact or non-EEG data, which may be a way to look for outliers of the data. In general, if the electrophysiology database and customized TMS treatment system 200 may artifact appropriately, there may not be many outliers because the outlier removal may be done on the front end (where if the overall data may not be clean enough, some data may be removed or data may be changed in terms of analysis in order to get metrics). Some of these metrics may be relatively robust. Accordingly, in some examples, there may be an attempt to try to remove outlier data that may not be related to brain activity. Therefore, there may be a need to limit the number of outliers. In some examples, it may be discovered that there are some populations that occur with relatively lower frequency that may be clustered appropriately (e.g., by artifacting data where some patient or brain types may have a lower population due to artifacting).

The electrophysiology database and customized TMS treatment system 200 (specifically the reporting system 206) may provide some statistics and may provide areas where improvement could be made. Some statistics may relate to identifying normative for each patient. In examples, the electrophysiology database and customized TMS treatment system 200 may utilize a number of databases and place a density of different EEG spectrums (e.g., density of theta) such that if more than the average, then there may be a combination of relative power measurements and normative database utilization determinations. The same approach may be used in finding neighbor or related brains that may be analyzed for differences. In some examples, the data may be aggregated that may involve time series data such that the electrophysiology database and customized TMS treatment system 200 may perform principal component analysis on the data, and the electrophysiology database and customized TMS treatment system 200 may perform coherence statistics. This may be done from narrow or wide-band perspectives in order to populate the database (of the electrophysiology database and customized TMS treatment system 200) to then feed into what may be considered to be normative, and the electrophysiology database and customized TMS treatment system 200 may determine how off the data may be in terms of standardizations from function. The electrophysiology database and customized TMS treatment system 200 may include components for identifying frequency relative to frequency preferences such as ideal frequency (e.g., zero versus present frequency).

The electrophysiology database and customized TMS treatment system 200 (using the reporting system 206) may generate a report that may indicate patients or individuals who have EEG characteristics that may be suboptimal compared to other more optimal EEG data (e.g., from a performance or general wellness standpoint optimal vs. suboptimal). In some examples, patients may have an opportunity to get therapy (e.g., NEST therapy) in a storefront and/or lease a NEST system to take home. The patient's optimal EEG may be compared to the population's optimal EEG and/or the patient's optimal EEG may be compared to the patient's actual EEG. In some of these examples, when the patient's optimal EEG may be compared to the patient's actual EEG, the results may indicate that the patient's EEG may not be suboptimal. Whereas, when the patient's optimal EEG may be compared to the population's optimal EEG, the results may indicate that the patient's actual EEG may be suboptimal. For example, EEGs of a patient's brain may have frequencies at about 10.5 Hertz, where the average for the patient's age may be about 9.7 Hz or about 10 Hz. This example may be great compared to the average, but the patient may also have an attenuated function or less that may be lower than an expected function for them at about 11.5 Hz and at about 12.3 Hz. Accordingly, the same patient may be suboptimal compared to themselves, but not to the average population. This is an example for frequency, which may also be similarly done for density of state activity where any amount of data may be compared. The patient's average amount versus the population may be optimal versus the population. Where the patient may have a lower amount versus the population, it may still be optimal. However, compared to the patient's own brain type, the average amount for the population may still be more than what may be expected which may be considered suboptimal. Thus, these comparisons may look at two dimensions (e.g., patient's optimal EEG versus population's optimal EEG or patient's optimal EEG versus patient's actual EEG).

The electrophysiology database and customized TMS treatment system 200 (using the reporting system 206) may generate a current brain profile (e.g., that may show chaotic aspects) and an optimal brain profile (minimal chaos to not being chaotic) for each patient, and a current or a generic brain profile. This may vary from one mental disorder or condition to another mental disorder or condition as well as based on each patient.

Electrophysiology Database and Customized TMS Treatment System—Modelling Treatments

The electrophysiology database and customized TMS treatment system 200 may use the treatment modelling system 220 to apply the electrophysiology database (e.g., Big Data from the patient recorded EEGs data store 208) to model treatments. The electrophysiology database and customized TMS treatment system 200 may include the electrophysiology database system that may apply big data analytics and/or aggregated data analytics on the EEGs (e.g., using the treatment modelling system 220, the electrophysiology analysis system 204, and/or the diagnosis and treatment system 210 in any combination) for determining the diagnosis and the treatment of each patient (e.g., providing electrophysiology big data analytics and/or aggregated data analytics) with respect to modelling treatments. The electrophysiology database system may apply the big data analytics and/or aggregated data analytics to model various treatments (e.g., using the treatment modelling system 220) in terms of predicted impacts to the patients and results of the treatments. In some examples, the treatment modelling system 220 may also utilize the predictive analytics system 222 in combination for providing predictive analytics of the EEG data and other data as described in the disclosure.

This may include model simulations of treatments before applied to patients (e.g., based on types of stimulation treatments, frequencies, location, etc.). In some examples, big data analytics and/or aggregated data analytics may be applied in order to model impacts of specific theta-burst stimulation (TBS) treatments and general TMS treatments (e.g., using the treatment modelling system 220). The treatment modelling system 220 may facilitate large-scale experiments with collection of substantial quantities of data. These large-scale experiments may try different factors in the application of TMS treatments, such as variation in the type and nature of the TMS device, alteration in magnet positions, variability in different energy levels, etc. The treatment modelling system 220 may provide for this data to be collected into centralized or decentralized database of most (if not all) experiments. The treatment modelling system 220 may use a depth of data that may allow for discovery of other potential experiments and for a creation of models that may predict likely outcomes of other dependent variables (e.g., predictions on usage patterns of different demographics without having actually conducted experiments on the different demographics or patient types). The treatment modelling system 220 may use adjustable factors such as electrode position, energy levels, duration of treatment, and similar patient responses as part of modelling for best treatment results for each patient such that these factors may be adjusted accordingly for each patient.

The treatment modelling system 220 may be used to look at new patients coming in that may be compared to other patients who may have also come in compared to treatments that current and old patients may have received. The treatment modelling system 220 may determine for those who may have received the treatments that are similar to those who are coming in, what may be the best treatment for them and adjust treatments accordingly.

The electrophysiology database and customized TMS treatment system 200 may be used to look at areas that may be dysfunctional in treating some patients (e.g., using the treatment modelling system 220). This may relate to areas that may make ideal function and reinforcing the function. Treating autism may produce a relatively large amount of posterior alpha frequencies and a relatively small amount of frontal alpha frequencies. Initially, superior frontal function was treated, as the frontal lobe may be where there may be a deficit, which may be treated. Then, when looking into circuitry, it was found that prefrontal might have better anterior to posterior connectivity than superior frontal stimulation or superior frontal circuitry. Treatment of the cortical tissue frontally may be adjusted accordingly.

In EEG data, the electrophysiology database and customized TMS treatment system 200 may have corroborative evidence that after adding posterior location and reinforcing posterior function, the reinforcement of global corresponding oscillations may be facilitated and observed. There may be a propagation of activity from posterior to anterior regions. For some patients, addition of a posterior location may facilitate function globally too quickly for safe clinical practice. Thus, when taking a sequential EEG, the electrophysiology database and customized TMS treatment system 200 may observe the degree to which this dysfunction of interest may be shifting in density and presence and then the treatment recommendation (e.g., execution by the portable NEST system 100) may be adjusted in terms of location, frequency, and other parameters in order to reinforce function, both from the source of function and where the function is observed to be at deficit or may have disruption.

Examples of Uses of Systems and Processes

There may be a variety of examples of uses and use cases involving the EEG and TMS process 400, the portable NEST system 100, and the electrophysiology database and customized TMS treatment system 200. These systems and processes may be used in a clinic-to-clinic basis. (e.g., TMS clinics). These clinics themselves may have TMS systems (e.g., portable NEST system 100) that may be using the same protocols and mechanisms described in the disclosure. These systems and processes may be widely accepted and used by the medical community such as at doctors' offices and hospitals.

In some examples, these systems and processes may be used in a NEST storefront (e.g., may obtain consumer data on how each patient may want to use their portable NEST system 100). A general concept of the storefront may include a person going in to have their EEG recorded. The recorded EEG may be uploaded to servers, and the electrophysiology database and customized TMS treatment system 200 may generate a report based on the EEG data that may point out characteristics of the EEG data related to each patient. This report may also provide some statistics and may provide areas where improvement could be made.

These systems and processes may be used in various portable use-case options (e.g., specifically using the portable NEST system 100). Including some of the examples described in the disclosure, other portable use-case options may also include combining the portable NEST system 100 into first-class airline seats of airplanes, using the portable NEST system 100 while sleeping at night, using the portable NEST system 100 while undergoing a test, and using the portable NEST system 100 while performing an activity requiring focus and/or concentration (e.g., race car driver, truck driver, virtual reality).

These systems and processes may be used with patients that may have persistent post-concussion syndrome and post-traumatic stress. This may relate to patients that may have been the most repeatable and interested from clinical indications.

These systems and processes may be used with military operators (e.g., especially special operations community such as elite operator seal teams and delta forces), athletes, and corporate executives. For these three categories, the systems and processes may use brain wave patterns to reach soldiers, athletes, and/or executives that may be deemed most likely prospective patients based on their brain conditions (e.g., using machine learning, pattern matching patient identification, etc.) such that these categories of people with high stress, high skill jobs may be most in need of a way to remain calm and focused in powerful lifestyles. For athletes, in some examples, athletes may be looking for treatment of not only head injuries but also for improvement performance. The systems and processes may be used to treat athletes in high stress situations such those athletes performing in the super bowl or the World Series. The system and processes may be also be useful to extreme sports athletes (e.g., surfing athletes and biking athletes). Corporate executives (e.g., CEOs) may need to utilize the systems and processes for improving memory, decision-making, improving sleep (e.g., if they may be jet lagged), etc.

These systems and processes may be used in the wellness industry. For example, within wellness industry, these systems and processes may be used with people towards improving focus, attention, concentration, potentially certain elements of sleep, etc. These systems and processes may be used at wellness businesses such as spas (e.g., kiosks where a person may get a massage may also include NEST treatment in combination).

As described, these systems and process may be used in treatment of psychological and neurological disorders. This may include treatment of autism, PTSD, mood disorders (e.g., depression), anxiety, ADHD, schizophrenia, etc. In some examples, the systems and processes may be used to determine patient mood and control delivery of therapy based on determined state. The systems and processes may be used to model brain dynamics in normal and diseased states for guiding therapy.

These systems and processes may be used for treatment of a variety of other medical issues and/or conditions. These systems and processes may be used for treatment of airway diseases or conditions (e.g., treatment of asthma, chronic obstructive pulmonary disease (COPD), allergies, etc.). These systems and processes may be used for treatment of seizures (e.g., epileptic seizures), headaches and other pain-related ailments (e.g., chronic pain), and urologic disorders. Urologic disorders may refer to neurologic bladder dysfunction such as patients with Parkinson's disease and other neurodegenerative disorders. These systems and process may also be used to treat addiction such as nicotine addiction or other drug addictions.

While only a few embodiments of the disclosure have been shown and described, it will be obvious to those skilled in the art that many changes and modifications may be made thereunto without departing from the spirit and scope of the disclosure as described in the following claims.

The methods and systems described herein may be deployed in part or in whole through a machine, that executes computer software, program codes, and/or instructions on a processor. The disclosure may be implemented as a method on the machine, as a system or apparatus as part of or in relation to the machine, or as a computer program product embodied in a computer readable medium executing on one or more of the machines. In embodiments, the processor may be part of a server, cloud server, client, network infrastructure, mobile computing platform, stationary computing platform, or other computing platforms. A processor may be any kind of computational or processing device capable of executing program instructions, codes, binary instructions and the like, including a central processing unit (CPU), a general processing unit (GPU), a logic board, a chip (e.g., a graphics chip, a video processing chip, a data compression chip, or the like), a chipset, a controller, a system-on-chip (e.g., an RF system on chip, an AI system on chip, a video processing system on chip, or others), an integrated circuit, an application specific integrated circuit (ASIC), a field programmable gate array (FPGA), an approximate computing processor, a quantum computing processor, a parallel computing processor, a neural network processor, or other type of processor. The processor may be or may include a signal processor, digital processor, data processor, embedded processor, microprocessor or any variant such as a co-processor (math co-processor, graphic co-processor, communication co-processor, video co-processor, AI co-processor, and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more threads. The thread may spawn other threads that may have assigned priorities associated with them; the processor may execute these threads based on priority or any other order based on instructions provided in the program code. The processor, or any machine-utilizing one, may include non-transitory memory that stores methods, codes, instructions and programs as described herein and elsewhere. The processor may access a non-transitory storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache, network-attached storage, server-based storage, and the like.

A processor may include one or more cores that may enhance speed and performance of a multiprocessor. In embodiments, the process may be a dual core processor, quad core processors, other chip-level multiprocessor and the like that combine two or more independent cores (sometimes called a die).

The methods and systems described herein may be deployed in part or in whole through a machine, that executes computer software on a server, client, firewall, gateway, hub, router, switch, infrastructure-as-a-service, platform-as-a-service, or other such computer and/or networking hardware or system. The software may be associated with a server that may include a file server, print server, domain server, internet server, intranet server, cloud server, infrastructure-as-a-service server, platform-as-a-service server, web server, and other variants such as secondary server, host server, distributed server, failover server, backup server, server farm, and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, social networks, and the like. Additionally, this coupling and/or connection may facilitate remote execution of programs across the network. The networking of some or all of these devices may facilitate parallel processing of a program or method at one or more locations without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs, or codes as described herein and elsewhere may be executed by the client. In addition, other devices required for the execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of programs across the network. The networking of some or all of these devices may facilitate parallel processing of a program or method at one or more locations without deviating from the scope of the disclosure. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements. The methods and systems described herein may be adapted for use with any kind of private, community, or hybrid cloud computing network or cloud computing environment, including those which involve features of software as a service (SaaS), platform as a service (PaaS), and/or infrastructure as a service (IaaS).

The methods, program codes, and instructions described herein and elsewhere may be implemented on a cellular network with multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, 4G, 5G, LTE, EVDO, mesh, or other network types.

The methods, program codes, and instructions described herein and elsewhere may be implemented on or through mobile devices. The mobile devices may include navigation devices, cell phones, mobile phones, mobile personal digital assistants, laptops, palmtops, netbooks, pagers, electronic book readers, music players and the like. These devices may include, apart from other components, a storage medium such as flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g., USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, network-attached storage, network storage, NVME-accessible storage, PCIE connected storage, distributed storage, and the like.

The methods and systems described herein may transform physical and/or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable code using a processor capable of executing program instructions stored thereon as a monolithic software structure, as standalone software modules, or as modules that employ external routines, code, services, and so forth, or any combination of these, and all such implementations may be within the scope of the disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices, artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps associated therewith, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable devices, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions. Computer software may employ virtualization, virtual machines, containers, dock facilities, and other capabilities.

Thus, in one aspect, methods described above and combinations thereof may be embodied in computer executable code that, when executing on one or more computing devices, performs the steps thereof. In another aspect, the methods may be embodied in systems that perform the steps thereof and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the disclosure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein might be employed in practicing the invention. It is intended that the claims define the scope of the invention and that methods and structures within the scope of these claims and their equivalents be covered thereby.

1. A system for recommending transcranial magnetic stimulation (TMS) comprising:
an electrophysiology database and customized TMS treatment system operable to access electroencephalogram (EEG) data for a patient, wherein the electrophysiology database and customized TMS treatment system is operable to classify the patient with a particular brain type based on the EEG data, and the electrophysiology database and customized TMS treatment system is operable to determine a synchronized transcranial magnetic stimulation (sTMS) treatment based on the classified brain type and the EEG data; and
wherein the electrophysiology database and customized TMS treatment system is operable to recommend the TMS treatment.

2. The system of claim 1 wherein the EEG data includes one or more EEGs from one or more of pre-treatments or post-treatments to provide diagnostic feedback and treatment feedback for the patient.

3. The system of claim 1 wherein the particular brain type is at least one of a highly rhythmic brain, a less rhythmic brain, a low energy brain, or a high energy brain.

4. The system of claim 1 wherein the particular brain type is at least one of an absorber-type of personality or an emitter-type of personality.

5. The system of claim 1 further comprising a portable neuro-electroencephalogram synchronization therapy (NEST) system that is operable to capture the EEG data from the patient for access by the electrophysiology database and customized TMS treatment system, and wherein the portable NEST system is operable to recommend the sTMS treatment to the patient.

6. A system for recommending transcranial magnetic stimulation (TMS) comprising:
an electrophysiology database and customized TMS treatment system operable to access electroencephalogram (EEG) data for a patient, wherein the electrophysiology database and customized TMS treatment system is operable to analyze the EEG data to determine a brain health indication of the patient, and the electrophysiology database and customized TMS treatment system is operable to determine synchronous TMS (sTMS) treatment based on the brain health indication and the EEG data; and
wherein the electrophysiology database and customized TMS treatment system is operable to recommend the sTMS treatment.

7. The system of claim 6 wherein the EEG data includes one or more EEGs from one or more of pre-treatments or post-treatments to provide diagnostic feedback and treatment feedback for the patient.

8. (canceled)

9. The system of claim 6 wherein the electrophysiology database and customized TMS treatment system uses the brain health indication for diagnosing the patient.

10. The system of claim 6 further comprising a portable neuro-electroencephalogram synchronization therapy (NEST) system operable to capture the EEG data from the patient for access by the electrophysiology database and customized TMS treatment system, and wherein the portable NEST system is operable to recommend sTMS to the patient.

11. A system for recommending transcranial magnetic stimulation (TMS) comprising:
an electrophysiology database and customized TMS treatment system operable to access electroencephalogram (EEG) data for a patient, wherein the electrophysiology database and customized TMS treatment system is operable to analyze the EEG data to compute bursting EEG data analysis to determine a synchronous TMS (sTMS) treatment; and
wherein the electrophysiology database and customized TMS treatment system is operable to recommend the sTMS treatment.

12. The system of claim 11 wherein the electrophysiology database and customized TMS treatment system is operable to provide a diagnosis for the patient using the bursting EEG data analysis.

13. The system of claim 11 wherein the electrophysiology database and customized TMS treatment system is operable to use the bursting EEG data analysis to determine one or more treatment parameters, wherein the treatment parameters comprise one or more of a stimulation component position, a magnetic field characteristic, an output waveform, a magnetic field spatial distribution, a magnetic field strength gradient, a device compatibility, or a safety feature.

14. The system of claim 11 wherein the EEG data comprises one or more EEGs from one or more of pre-treatments or post-treatments to provide diagnostic feedback and treatment feedback for the patient.

15. The system of claim 11 further comprising a portable neuro-electroencephalogram synchronization therapy (NEST) system operable to capture the EEG data from the patient for access by the electrophysiology database and customized TMS treatment system, and wherein the portable NEST system is operable to recommend the TMS to the patient.

16. The system of claim 11, wherein the electrophysiology database and customized TMS treatment system comprises normative EEG data used to compute the bursting EEG data analysis.

17. The system of claim 11, wherein the electrophysiology database and customized TMS treatment system is operable to compute the bursting EEG data analysis using one or more of an artificial intelligence algorithm or a machine learning algorithm.

18. The system of claim 11, wherein the electrophysiology database and customized TMS treatment system comprises a treatment modeling system comprising one or more adjustable factors including electrode position, energy levels, or duration of treatment.

19. The system of claim 11, wherein the electrophysiology database and customized TMS treatment system is operable to recommend the sTMS treatment based on a condition comprising one or more of post-traumatic stress disorder (PTSD), depression, anxiety, autism spectrum disorder, Alzheimer's, traumatic brain injury, or generalized anxiety disorder.

20. The system of claim 11, wherein the electrophysiology database and customized TMS treatment system is operable to recommend the sTMS treatment based on a neurotransmitter density change.

21. The system of claim 11, wherein the electrophysiology database and customized TMS treatment system is operable to recommend the sTMS treatment based on a neuronal connectivity.
